(19) United States
(12) Patent Application Publication
(10) Pub. No.: US 2007/0161513 A1
(43) Pub. Date: Jul. 12, 2007

(54) 3-[1-halo-1-arylmethanesulfonyl]- and 3-[1-halo-1-heteroarylmethanesulfonyl]- isoxazoline derivatives, processes for preparing them, and use as herbicides and plant growth regulators

(75) Inventors: Hendrik Helmke, Liederbach (DE); Wolfgang Schaper, Diedorf (DE); Thomas Auler, Bad Soden (DE); Heinz Kehne, Hofheim (DE); Martin Hills, Idstein (DE); Dieter Feucht, Eschborn (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience GmbH, Frankfurt (DE)

(21) Appl. No.: 11/478,718

(22) Filed: Jul. 3, 2006

(30) Foreign Application Priority Data
Jul. 6, 2005 (DE) 102005031412.0

(51) Int. Cl. A01N 43/60; A01N 43/58; A01N 43/54; A01N 43/80; C07D 413/02

(52) U.S. Cl. 504/235; 504/271; 548/221; 544/238; 544/333; 544/405; 546/272.1; 548/182; 504/236; 504/239

(57) ABSTRACT

3-[1-Halo-1-arylmethanesulfonyl]- and 3-[1-halo-1-heteroarylmethanesulfonyl]-isoxazoline derivatives, processes for preparing them, and use as herbicides and plant growth regulators
The present invention provides compounds of the formula (I) and salts thereof, 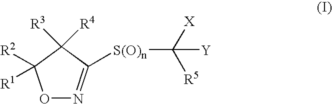 processes for preparing them, and their use as herbicides and plant growth regulators, particularly as herbicides for selectively controlling weed plants in crops of useful plants.

3-[1-Halo-1-arylmethanesulfonyl]- and 3-[1-halo-1-heteroarylmethanesulfonyl]-isoxazoline derivatives, processes for preparing them, and use as herbicides and plant growth regulators

The invention relates to the technical field of crop protection compositions, such as herbicides and plant growth regulators, particularly of herbicides for selective control of weed plants in crops of useful plants.

From various publications it is already known that certain isoxazoline derivatives possess herbicidal properties. For instance, patents JP-Δ-9328477, JP-Δ-9328483, WO 2001012613, WO 2002062770, WO 2003000686, WO 2004014138 and JP 2005-035924 describe isoxazoline derivatives which carry a heteroarylmethylsulfonyl group or an arylmethylsulfonyl group as a substituent in position 3 of the isoxazoline ring.

When applied, however, the active compounds already known from the publications identified above exhibit disadvantages, be it (a) an absent or inadequate herbicidal effect against weed plants, (b) an excessively narrow spectrum of weed plants that can be controlled with an active compound, or (c) insufficient selectivity in crops of useful plants.

It is therefore desirable to provide alternative active chemical compounds which can be employed where appropriate with advantages as herbicides or plant growth regulators.

The present invention provides compounds of the formula (I) and salts thereof 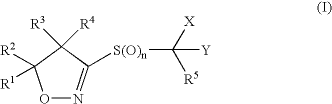
in which
R¹ and R² independently of one another are each H, cyano, (C₁-C₆)-alkyl, (C₂-C₆)-alkenyl, (C₂-C₆)-alkynyl, (C₃-C₈)-cycloalkyl, (C₁-C₆)-alkanoyl, (C₁-C₆)-alkoxycarbonyl, mono-((C₁-C₆)-alkyl)-aminocarbonyl, di-((C₁-C₆)-alkyl)-aminocarbonyl, each of the (C₁-C₆)-alkyl, (C₂-C₆)-alkenyl, (C₂-C₆)-alkynyl, (C₃-C₆)-cycloalkyl, (C₁-C₆)-alkanoyl, (C₁-C₆)-alkoxycarbonyl, mono-((C₁-C₆)-alkyl)-aminocarbonyl, di-((C₁-C₆)-alkyl)-aminocarbonyl radicals being unsubstituted or substituted by one or more identical or different radicals from the group consisting of halogen, cyano, and (C₃-C₈)-cycloalkyl or else by —OR⁶ or —S(O)ₘR⁶, in which m is the number 0, 1 or 2, R⁶ is a (C₁-C₆)-alkyl, (C₂-C₆)-alkenyl, (C₂-C₆)-alkynyl or (C₃-C₆)-cycloalkyl which is unsubstituted or substituted by one or more identical or different radicals from the group consisting of halogen and cyano,
   or independently of one another are each unsubstituted or substituted phenyl or heterocyclyl, of which the heterocyclyl can be saturated or unsaturated and contains preferably one or more, in particular 1, 2 or 3 heteroatoms in the heterocyclic ring, preferably from the group consisting of N, O, and S, and is preferably an aliphatic heterocyclyl radical having 3 to 7 ring atoms or a heteroaromatic radical having 5 or 6 ring atoms, and each of the aforementioned radicals being unsubstituted or substituted if desired by one or more identical or different radicals from the group consisting of (C₁-C₆)-alkyl, (C₁-C₆)-haloalkyl, halogen, cyano, and (C₃-C₈)-cycloalkyl, or —OC(R⁷)₃, or else —SC(R⁷)₃, R⁷ in the two last-mentioned groups being independently at each occurrence H, F or Cl,
   or else R¹+R² together form a spirolinkage of 3 to 7 carbon atoms, together with the carbon atom to which they are attached in unison,
R³ and R⁴ are H, (C₁-C₆)-alkyl, (C₂-C₆)-alkenyl, (C₂-C₆)-alkynyl or (C₃-C₈)-cycloalkyl, the aforementioned alkyls, cycloalkyls, alkenyls or alkynyls being substituted if desired by one or more identical or different radicals from the group consisting of halogen, nitro, cyano, (C₃-C₆)-cycloalkyl, (C₁-C₆)-alkoxy, (C₁-C₆)-haloalkoxy, and (C₁-C₆)-alkylthio, or else R³+R⁴ together form a spirolinkage of 3 to 7 carbon atoms, together with the carbon atom to which they are attached in unison, or else
R¹+R³ form, together with the carbon atoms to which they are attached, a ring structure composed of 5-8 carbon atoms,
R⁵ is unsubstituted or substituted aryl, preferably having 6 to 14 carbon atoms, or unsubstituted or substituted heteroaryl, having preferably 1 to 9 carbon atoms and one or more heteroatoms, preferably having 1 to 4 heteroatoms, in particular having 1 to 4 heteroatoms from the group consisting of N, O, and S, each of the above carbocyclic or heterocyclic radicals being substituted if desired by OH, halogen, cyano, (C₁-C₆)-alkyl, (C₁-C₆)-haloalkyl, (C₂-C₆)-alkenyl, (C₂-C₆)-alkynyl, (C₃-C₆)-cyclo-alkyl, (C₃-C₆)-cycloalkenyl, mono-(C₁-C₆)-alkylamino, di-((C₁-C₆)-alkyl)-amino, N—((C₁-C₆)-alkanoyl)-amino, (C₁-C₆)-alkoxy, (C₁-C₆)-haloalkoxy, (C₃-C₆)-alkenyloxy, (C₃-C₆)-alkynyloxy, (C₃-C₆)-cycloalkoxy, (C₄-C₆)-cycloalkenyloxy, (C₁-C₆)-alkylthio, (C₁-C₆)-haloalkylthio, (C₃-C₆)-cyclo-alkylthio, (C₃-C₆)-alkenylthio, (C₄-C₆)-cycloalkenylthio, (C₃-C₆)-alkynyl-thio, (C₁-C₆)-alkanoyl, (C₂-C₆)-alkenylcarbonyl, (C₂-C₆)-alkynylcarbonyl, arylcarbonyl, (C₁-C₆)-alkoxycarbonyl, (C₃-C₆)-alkenyloxycarbonyl, (C₃-C₆)-alkynoxycarbonyl, aryloxycarbonyl, (C₁-C₆)-alkylsulfinyl, (C₁-C₆)-alkylsulfonyl, (C₁-C₆)-haloalkylsulfinyl or (C₁-C₆)-haloalkylsulfonyl, and aforementioned alkyl, alkoxy or haloalkoxy radicals being, if desired, linked cyclically with one another, with the proviso that they are in ortho position,
   n is 1, or 2,
   X is F, Cl, Br or I, and
   Y is H, F, Cl, Br or I.

Preferred compounds of the formula (I) are those wherein R¹ and R² independently of one another are each H, (C₁-C₄)-alkyl, (C₂-C₃)-alkenyl, (C₂-C₃)-alkynyl or (C₃-C₆)-cycloalkyl, each of the (C₁-C₄)-alkyl, (C₂-C₃)-alkenyl, (C₂-C₃)-alkynyl and (C₃-C₆)-cycloalkyl radicals being substituted if desired by one or more radicals from the group consisting of halogen, (C₁-C₃)-alkoxy, cyano, and (C₃-C₆)-cycloalkyl.

Particularly preferred compounds of the formula (I) are those wherein R¹ and R² independently of one another are (C₁-C₄)-alkyl, each of the (C₁-C₄)-alkyl radicals being substituted if desired by one or more identical or different halogens, preferably by chlorine, bromine or fluorine.

Particularly preferred compounds of the formula (I) are also those wherein R¹ and R² independently of one another are methyl or ethyl which in turn may, if desired, independently of one another, be monohalogenated or polyhalogenated, preferably chlorinated or fluorinated. Among the halogenated radicals particular preference is given in this context to chloromethyl and fluoromethyl, especially chloromethyl.

Further-preferred compounds of the formula (I) are those wherein R³ and R⁴ are H or (C₁-C₄)-alkyl, the alkyl being substituted if desired by one or more identical or different radicals from the group consisting of halogen and cyano.

Particularly preferred compounds of the formula (I) are those wherein R³ and R⁴ are an H, methyl or ethyl.

Very particularly preferred compounds of the formula (I) are those wherein R³ and R⁴ are an H.

Further-preferred compounds of the formula (I) are those wherein R⁵ is an unsubstituted or substituted aryl, preferably having 6 to 10 carbon atoms, or unsubstituted or substituted heteroaryl, having preferably 1 to 9 carbon atoms, preferably 2 to 5 carbon atoms, having 1 to 3 heteroatoms from the group consisting of N, O and S, each of the above carbocyclic or heterocyclic radicals being substituted if desired by OH, halogen, cyano, (C₁-C₄)-alkyl, (C₁-C₄)-haloalkyl, (C₂-C₄)-alkenyl, (C₂-C₄)-alkynyl, (C₃-C₆)-cycloalkyl, (C₅-C₆)-cycloalkenyl, mono-(C₁-C₄)-alkylamino, di-((C₁-C₄)-alkyl)-amino, N—((C₁-C₄)-alkanoyl)-amino, (C₁-C₄)-alkoxy, (C₁-C₄)-haloalkoxy, (C₃-C₄)-alkenyloxy, (C₃-C₄)-alkynyloxy, (C₄-C₆)-cycloalkoxy, (C₅-C₆)-cycloalkenyloxy, (C₁-C₄)-alkylthio, (C₁-C₄)-haloalkylthio, (C₄-C₆)-cycloalkyl-thio, (C₃-C₄)-alkenylthio, (C₅-C₆)-cycloalkenylthio, (C₃-C₄)-alkynylthio, (C₁-C₄)-alkanoyl, (C₂-C₄)-alkenylcarbonyl, (C₂-C₄)-alkynylcarbonyl, arylcarbonyl, (C₁-C₄)-alkoxycarbonyl, (C₃-C₄)-alkenyloxycarbonyl, (C₃-C₄)-alkynoxycarbonyl, aryloxy-carbonyl, (C₁-C₄)-alkylsulfinyl, (C₁-C₄)-alkylsulfonyl or (C₁-C₄)-haloalkylsulfinyl or (C₁-C₄)-haloalkylsulfonyl, and aforementioned alkyl, alkoxy, and haloalkoxy radicals being, if desired, linked cyclically with one another, with the proviso that they are in ortho position.

Preferred compounds of the formula (I) are also those wherein R⁵ is an unsubstituted or substituted aryl, preferably having 6 to 10 carbon atoms, or unsubstituted or substituted heteroaryl, having preferably 2 to 5 carbon atoms with 1 to 3 heteroatoms from the group consisting of N, O, and S, each of the above carbocyclic or heterocyclic radicals being substituted if desired by one or more identical or different radicals from the group consisting of halogen, cyano, ethyl, methyl, haloethyl, halomethyl, methoxy, ethoxy, trifluoromethylthio, trifluoromethylsulfinyl, trifluoromethylsulfonyl, halomethoxy, and haloethoxy.

Particularly preferred compounds of the formula (I) are those wherein R⁵ is a substituted or unsubstituted phenyl, pyrazolyl, pyridinyl, pyrimidinyl, pyrazinyl, pyridazinyl, thienyl, furyl, imidazolyl, triazolyl, isothiazolyl, thiazolyl or oxazolyl, very preferably a phenyl or pyrazole, which if substituted are substituted preferably by one or more identical or different radicals from the group consisting of halogen, cyano, ethyl, methyl, methoxy, ethoxy, halomethoxy, haloethoxy, trifluoromethylthio, trifluoromethylsulfinyl, trifluoromethylsulfonyl, haloethyl, and halomethyl, preference among the halogens being given to chlorine and fluorine, especially fluorine.

Particularly preferred compounds of the formula (I) are those wherein R⁵ is a phenyl or pyrazole bearing one, two or three, preferably one or two, identical or different substituents from the group consisting of fluorine, chlorine, methyl, trifluoromethyl, methoxy, difluoromethoxy, and trifluoromethoxy.

Further-preferred compounds of the formula (I) are those wherein X is a chlorine, fluorine or bromine, more preferably a chlorine or fluorine.

Preferred compounds of the formula (I) are also those wherein Y is an H, chlorine, fluorine or bromine, more preferably an H, chlorine or fluorine, very preferably an H.

Particularly preferred compounds of the formula (I) are those wherein X is a chlorine and Y is an H.

Particularly preferred compounds of the formula (I) are also those wherein X is a bromine and Y is an H.

Additionally particularly preferred compounds of the formula (I) are those wherein X is a fluorine and Y is an H.

Further-preferred compounds of the formula (I) are those wherein n is 2.

The compounds of the formula (I) can form salts by addition of a suitable organic or inorganic acid, such as HCl, HBr, H₂SO₄ or HNO₃, for example, or else oxalic acid or sulfonic acids, onto a basic group, such as amino or alkylamino. Suitable substituents present in deprotonated form, such as sulfonic acids or carboxylic acids, can form inner salts with groups which can themselves be protonated, such as amino groups. Salts may likewise be formed by the replacement of hydrogen in suitable substituents, such as sulfonic acids or carboxylic acids, by an agriculturally suitable cation. These salts are, for example, metal salts, especially alkali metal salts or alkaline earth metal salts, particularly sodium salts and potassium salts, or else ammonium salts, salts with organic amines or quaternary ammonium salts with cations of the formula [NRR′R″R′″]⁺, in which R to R′″ each independently are an organic radical, especially alkyl, aryl, aralkyl or alkylaryl.

Compounds of the invention of the formula (I) and their salts are also referred to for short below as “compounds (I) of the invention”.

In formula (I) and all formulae below, the radicals alkyl, alkoxy, haloalkyl, haloalkoxy, alkylamino, alkylthio, haloalkylthio, alkylsulfinyl, alkylsulfonyl, haloalkylsulfinyl and haloalkylsulfonyl, and also the corresponding unsaturated and/or substituted radicals, may in each case be straight-chain or branched in the carbon skeleton.

Unless specifically indicated, for these radicals the lower carbon skeletons, such as those having 1 to 6 carbon atoms, especially 1 to 4 carbon atoms, or, in the case of unsaturated groups, those with 2 to 6 carbon atoms, particularly 2 to 4 carbon atoms, are preferred. Alkyl radicals, both alone and in composite definitions such as alkoxy, haloalkyl, etc, are for example methyl, ethyl, n- or i-propyl, n-, i-, t- or 2-butyl, pentyls, hexyls, such as n-hexyl, i-hexyl and 1,3-dimethylbutyl, heptyls, such as n-heptyl, 1-methylhexyl and 1,4-dimethylpentyl; alkenyl and alkynyl radicals have the definition of the possible unsaturated radicals corresponding to the alkyl radicals, there being at least one double bond or triple bond, preferably one double bond or triple bond, respectively. Alkenyl is for example vinyl, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is for example ethynyl, propargyl, but-2-yn-1-yl, but-3-yn-1-yl and 1-methylbut-3-yn-1-yl.

Halogen is fluorine, chlorine, bromine or iodine. Haloalkyl, haloalkenyl and haloalkynyl are alkyl, alkenyl or alkynyl substituted fully or partly by halogen, preferably by fluorine, chlorine or bromine, in particular by fluorine and/or chlorine, examples being monohaloalkyl (i.e. mono-halogen-alkyl), perhaloalkyl, CF₃, CHF₂, CH₂F, CF₃CF₂, CH₂FCHCl, CCl₃, CHCl₂, CH₂CH₂Cl; haloalkoxy is for example OCF₃, OCHF₂, OCH₂F, CF₃CF₂O, OCH₂CF₃, and OCH₂CH₂Cl; corresponding comments apply to haloalkenyl and other halogen-substituted radicals.

Aryl is a monocyclic, bicyclic or polycyclic aromatic system, phenyl or naphthyl for example, preferably phenyl.

A heterocyclic radical or ring (heterocyclyl or heteroaryl) can be saturated, unsaturated or heteroaromatic; unless defined otherwise, it contains preferably one or more, in particular 1, 2 or 3, heteroatoms in the heterocyclic ring, preferably from the group consisting of N, O, and S; preferably it is an aliphatic heterocyclyl radical having 3 to 7 ring atoms or a heteroaromatic radical having 5 or 6 ring atoms. The heterocyclic radical may for example be a heteroaromatic radical or ring (heteroaryl), such as monocyclic, bicyclic or polycyclic aromatic system in which at least one ring contains one or more heteroatoms. Preferably it is a heteroaromatic ring with a heteroatom from the group consisting of N, O and S, such as pyridyl, pyrrolyl, thienyl or furyl for example; with further preference it is a corresponding heteroaromatic ring having 2 or 3 heteroatoms, such as pyrimidinyl, pyridazinyl, pyrazinyl, triazinyl, thiazolyl, thiadiazolyl, oxazolyl, isoxazolyl, pyrazolyl, imidazolyl, and triazolyl. With further preference it is a partly or fully hydrogenated heterocyclic radical having a heteroatom from the group consisting of N, O, and S, examples being oxiranyl, oxetanyl, oxolanyl (=tetrahydrofuryl), oxanyl, pyrrolidyl (=pyrrolidinyl) or piperidyl or else pyrrolinyl such as Δ¹-pyrrolinyl, Δ²-pyrrolinyl or Δ³-pyrrolinyl, for example Δ¹-pyrrolin-2-yl, Δ¹-pyrrolin-3-yl, Δ¹-pyrrolin-4-yl or Δ¹-pyrrolin-5-yl or Δ²-pyrrolin-1-yl, Δ²-pyrrolin-2-yl, Δ²-pyrrolin-3-yl, Δ²-pyrrolin-4-yl, Δ²-pyrrolin-5-yl or Δ³-pyrrolin-1-yl, Δ³-pyrrolin-2-yl or Δ³-pyrrolin-3-yl.

With further preference it is a partly or fully hydrogenated heterocyclic radical having 2 heteroatoms from the group consisting of N, O, and S, examples being piperazinyl, dioxanyl, dioxolanyl, oxazolinyl, isoxazolinyl, oxazolidinyl, isoxazolidinyl, and morpholinyl.

Suitable substituents for a substituted heterocyclic radical include the substituents specified later on below, and also oxo as well. The oxo group may also occur on the hetero-ring atoms which are able to exist in different oxidation states, as for N and S, for example.

The definition “substituted by one or more radicals” refers, unless otherwise defined, to one or more identical or different radicals.

The exemplified substituents (“first substituent level”), where they contain hydrocarbon components, may if desired be further substituted in those components (“second substituent level”), as for example by one of the substituents as defined for the first substituent level. Corresponding further substituent levels are possible. The term “substituted radical” preferably encompasses only one or two substituent levels.

In the case of radicals containing carbon atoms, those having 1 to 6 carbon atoms, preferably 1 to 4 carbon atoms, in particular 1 or 2 carbon atoms, are preferred. Preference is generally given to substituents from the group consisting of halogen, fluorine and chlorine for example, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, nitro, and cyano.

Unsubstituted or substituted phenyl is preferably phenyl which is unsubstituted or substituted one or more times, preferably up to three times, by identical or different radicals from the group consisting of halogen, (C₁-C₄)-alkyl, (C₁-C₄)-alkoxy, (C₁-C₄)-haloalkyl, (C₁-C₄)-haloalkoxy, and nitro, such as o-, m-, and p-tolyl, dimethylphenyls, 2-, 3-, and 4-chlorophenyl, 2-, 3-, and 4-trifluoromethyl- and 2-, 3-, and 4-trichloro-methylphenyl, 2,4-, 3,5-, 2,5-, and 2,3-dichlorophenyl, o-, m-, and p-methoxyphenyl.

An acid radical of an inorganic or organic oxygen acid is a radical formed formally by removal of a hydroxyl group from the acid function, an example being the sulfo radical —SO₃H, derived from sulfuric acid H₂SO₄, or the sulfino radical —SO₂H, derived from sulfurous acid H₂SO₃, or, correspondingly, the group SO₂NH₂, the phospho radical —PO(OH)₂, the group —PO(NH₂)₂, —PO(OH)(NH₂), —PS(OH)₂, —PS(NH₂)₂ or —PS(OH)(NH₂), the carboxyl radical COOH, derived from carbonic acid, and radicals of the formula —CO—SH, —CS—OH, —CS—SH, —CO—NH₂, —CS—NH₂, —C(═NH)—OH or —C(═NH)—NH₂; additionally suitable are radicals with hydrocarbon radicals or substituted hydrocarbon radicals, i.e., acyl radicals in the wider sense (i.e. “acyl”).

The invention also provides all stereoisomers encompassed by formula (I), and mixtures thereof. Such compounds of the formula (I) contain one or more asymmetric carbon atoms (i.e. asymmetrically substituted carbon atoms), and/or asymmetric sulfur atoms in the form of sulfoxides, or else double bonds, which are not indicated specifically in the formulae (I). The possible stereoisomers defined by their specific three-dimensional form, such as enantiomers, diastereomers, Z-isomers, and E-isomers, are all encompassed by the formula (I) and can be obtained by standard methods from mixtures of the stereoisomers or else prepared by stereoselective reactions in combination with the use of stereochemically pure starting materials.

For the reasons in particular of greater herbicidal effect, improved selectivity and/or greater ease of preparation, particular interest attaches to compounds of the invention of the stated formula (I) or salts thereof in which individual radicals have one of the preferred definitions already specified or specified below, or, in particular, to those in which one or more of the preferred definitions already specified or specified below occur in combination.

The general definitions of radicals cited above, or those cited in ranges of preference, apply not only to the end products of the formula (I) but also, correspondingly, to the starting materials and intermediates required for the preparation. These definitions of radicals can be interchanged with one another, including interchange between the preferred ranges specified.

The present invention further provides methods of preparing the compounds of the formula (I) and/or their salts. Compounds of the formula (I) of the invention can alternatively be prepared by various analogous known methods.
a.) For the preparation of compounds of the formula (I′) in which R¹, R², R³, R⁴, and R⁵ have the definitions given in relation to the formula (I), n is 1 or 2, and X is fluorine 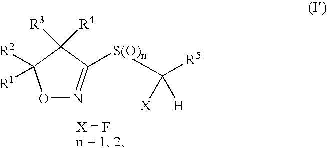
for example, a thioether of the formula (II) 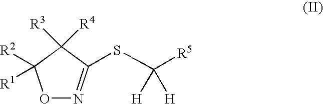
in which R¹, R², R³, R⁴, and R⁵ have the definitions given for formula (I) is fluorinated with an electrophilic fluorinating agent in the position alpha to the sulfur atom, and the resulting alpha-fluorothioether of the formula (III) 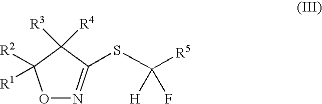
in which R¹, R², R³, R⁴, and R⁵ have the definitions indicated in relation to the formula (I) is oxidized with one equivalent of an oxidizing agent to give the fluorosulfoxides (I′) for which n is 1, or is oxidized with two equivalents of an oxidizing agent, to give the fluorosulfones (I′) for which n is 2. A suitable fluorinating agent for preparing the derivatives (III) is, for example, 1-chloromethyl-4-fluoro-1,4-diazabicyclo[2.2.2]octane bistetrafluoroborate (F-TEDA-BF₄, Selectfluor™). Examples of suitable oxidizing agents for preparing the sulfoxides (n=1) include hydrogen peroxide, sodium metaperiodate, organic peroxides, such as tert-butyl hydroperoxide, or organic peroxy acids, such as peracetic acid or, preferably, 3-chloroperbenzoic acid (J. Org. Chem. 58 (1993), 2791). Examples of suitable oxidizing agents for preparing the sulfones (n=2) include hydrogen peroxide, organic peroxides, such as tert-butyl hydroperoxide, or organic peroxy acids, such as peracetic acid or, preferably, 3-chloroperbenzoic acid. The preparation of the thioethers of the formula (II) is described for example in WO 2001 012613, WO 2002 062770, WO 2003 000686, and WO 2003 010165.
b.) A process for preparing compounds of the formula (I″), in which R¹, R², R³, R⁴, and R⁵ have the definitions indicated for the formula (I) and X is fluorine, chlorine or bromine 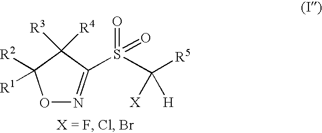
involves deprotonating a sulfone of the formula (IV), 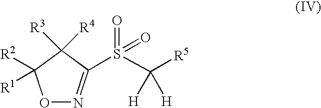
in which R¹, R², R³, R⁴, and R⁵ have the definitions indicated for the formula (I) in the position alpha to the sulfur atom and alternatively fluorinating the resulting carbanion with an electrophilic fluorinating agent, to give the fluorosulfones (I″) with X=fluorine, or chlorinating it with a chlorinating reagent, to give the chlorosulfones (I″) with X=chlorine, or brominating it with a brominating reagent, to give the bromosulfones (I″) with X=bromine.

For the preparation of the fluorosulfones the sulfones of the formula (IV) are deprotonated with a strong base, such as lithium or potassium diisopropylamide, lithium, sodium or potassium hexamethyl disilazane, n- or tert-butyllithium or potassium tert-butoxide, for example, in a suitable inert solvent, such as tetrahydrofuran, dioxane or dimethylformamide, for example, and then reacted with an electrophilic fluorinating agent such as, for example, 1-chloromethyl-4-fluoro-1,4-diazabicyclo[2.2.2]octane bistetrafluoroborate (F-TEDA-BF₄, Selectfluor™) or N-fluorobenzenesulfonimide (AccuFluor™).

For preparing the chlorosulfones or the bromosulfones, the sulfones of the formula (IV) are deprotonated preferably with caustic soda or caustic potash and then chlorinated or brominated with carbon tetrachloride or carbon tetrabromide (cf. R. R. Regis, A. M. Dowejko, Tetrahedron Lett. 23 (1982), 2539). The preparation of sulfones of the formula (IV) is described for example in WO 2001 012613, WO 2002 062770, WO 2003 000686, and WO 2003 010165.
c.) For preparing compounds of the formula (I′″) in which R¹, R², R³, R⁴, and R⁵ have the definitions indicated for the formula (I) and X is chlorine 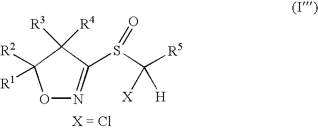
a sulfoxide of the formula (V) 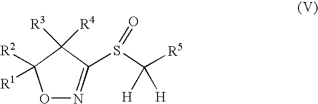
in which R¹, R², R³, R⁴, and R⁵ have the definitions indicated for the formula (I), is deprotonated in the position alpha to the sulfur atom, and the resultant carbanion is reacted with a halogenating agent, preferably carbon tetrachloride, to give the chlorosulfoxides (I′″). The reaction regime corresponds to the above-described preparation of the chlorosulfones (I″).

The preparation of sulfoxides of the formula (V) is described for example in WO 2001 012613, WO 2002 062770, WO 2003 000686, and WO 2003 010165.
d.) Another process for preparing the chlorosulfoxides of the formula (I′″) 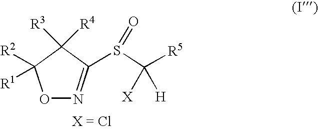
involves reacting a thioether of the formula (II) 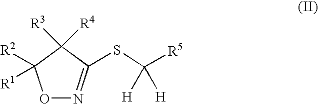
with elemental chlorine in dichloromethane/water in the presence of sodium dihydrogen phosphate.

The chlorosulfoxides (I′″) prepared according to c.) or d.) can if desired be oxidized with a suitable oxidizing agent, such as hydrogen peroxide, organic peroxides, such as tert-butyl hydroperoxide, or organic peroxy acids, such as peracetic acid or, preferably, 3-chloroperbenzoic acid, to give the chlorosulfones (I″) with X=chlorine.
e.) For preparing the compounds of the formula (I″″) for which R¹, R², R³, R⁴, and R⁵ have the definitions indicated for the formula (I), and X and Y are each chlorine, bromine or fluorine 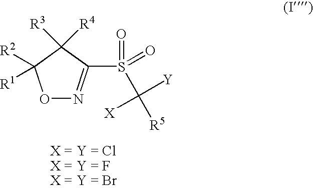
the dichlorosulfones are prepared in the same way as for the preparation of the monochlorosulfones (I″) in accordance with method b.), but the carbon tetrachloride halogenating agent is employed in excess.

For preparing the dibromosulfones the procedure is similar to that for preparing the monobromosulfones (I″) in accordance with method b.) but involves using the carbon tetrabromide halogenating agent in excess.

For preparing the difluorosulfones the procedure is again similar to that for preparing the monofluorosulfones (I″) in accordance with method b.), in terms of the bases and the fluorinating agents, but in each case twice the equivalent of the base and of the fluorinating agent is employed.

Techniques suitable for preparing enantiomers of the compounds (I) include not only enantioselective syntheses but also typical racemate separation methods (cf. handbooks of stereochemistry), such as by adduct formation with an optically active auxiliary reagent, separation of the diastereomeric adducts into the corresponding diastereomers, by crystallization, chromatographic methods, particularly column chromatography and high-pressure liquid chromatography, for example, distillation, where appropriate under reduced pressure, extraction, and other methods, and subsequent cleavage of the diastereomers back into the enantiomers. For preparative quantities or on the industrial scale, suitable techniques include those such as the crystallization of diastereomeric salts, which can be obtained from the compounds (I) using optically active acids and where appropriate, with acidic groups present, using optically active bases.

Optically active acids suitable for separating racemates by crystallization of diastereomeric salts include, for example, camphorsulfonic acid, camphoric acid, bromocamphorsulfonic acid, quinic acid, tartaric acid, dibenzoyltartaric acid, and other, analogous acids; suitable optically active bases include, for example, quinine, cinchonine, quinidine, brucine, 1-phenylethylamine, and other, analogous bases.

The crystallizations are then in most cases carried out in aqueous or aqueous-organic solvents, where the diastereomer which is less soluble precipitates first, if appropriate after seeding. One enantiomer of the compound of the formula (I) is then liberated from the precipitated salt, or the other is liberated from the crystals, by acidification or using a base.

Racemates can also be separated by chromatography on chiral columns.

The following acids are suitable for preparing the acid addition salts of the compounds of the formula (I): hydrohalic acids, such as hydrochloric acid or hydrobromic acid, furthermore phosphoric acid, nitric acid, sulfuric acid, mono- or bifunctional carboxylic acids and hydroxycarboxylic acids, such as acetic acid, maleic acid, succinic acid, fumaric acid, tartaric acid, citric acid, salicylic acid, sorbic acid or lactic acid, and also sulfonic acids, such as p-toluenesulfonic acid and 1,5-naphthalenedisulfonic acid. The acid addition compounds of the formula (I) can be obtained in a simple manner by the customary methods for forming salts, for example by dissolving a compound of the formula (I) in a suitable organic solvent, such as, for example, methanol, acetone, methylene chloride or benzine, and adding the acid at temperatures from 0 to 100° C., and they can be isolated in the known manner, for example by filtration, and, if appropriate, purified by washing with an inert organic solvent.

The base addition salts of the compounds of the formula (I) are preferably prepared in inert polar solvents, such as, for example, water, methanol or acetone, at temperatures from 0 to 100° C. Examples of bases which are suitable for the preparation of the salts according to the invention are alkali metal carbonates, such as potassium carbonate, alkali metal hydroxides and alkaline earth metal hydroxides, for example NaOH or KOH, alkali metal hydrides and alkaline earth metal hydrides, for example NaH, alkali metal alkoxides and alkaline earth metal alkoxides, for example sodium methoxide or potassium tert-butoxide, or ammonia, ethanolamine or a quarternary ammonium hydroxide of the formula [NRR′R″R′″]⁺ OH⁻.

Solvents referred to as “inert solvents” in the above process variants are to be understood as meaning in each case solvents which are inert under the reaction conditions in question, but which need not be inert under any reaction conditions.

A collection of compounds of the formula (I) which can be synthesized by the abovementioned process may also be prepared in a parallel manner where the process may be carried out manually, partially automated or fully automated. In this case, it is possible to automate the procedure of the reaction, the work-up or the purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Escom, 1997, pages 69 to 77.

A number of commercially available apparatuses as are offered by, for example, Stem Corporation, Woodrolfe Road, Tollesbury, Essex, CM9 8SE, England or H+P Labortechnik GmbH, Bruckmannring 28, 85764 Oberschleiβheim, Germany may be used for the parallel implementation of the reaction and work-up. For the parallel purification of compounds (I), or of intermediates obtained during the preparation, use may be made, inter alia, of chromatography apparatuses, for example those from ISCO, Inc., 4700 Superior Street, Lincoln, Nebr. 68504, USA. The apparatuses mentioned make possible a modular procedure in which the individual process steps are automated, but manual operations have to be performed between the process steps. This can be avoided by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to the methods described here, compounds of the formula (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products from IRORI, 11149 North Torrey Pines Road, La Jolla, Calif. 92037, USA, are employed, may be partially automated. The automation of solid-phase-supported parallel synthesis is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

Preparation by the methods described here yields compounds of the formula (I) in the form of collections of substances known as libraries. The present invention therefore also provides libraries of the compounds of the formula (I) which contain at least two compounds of the formula (I) and their intermediates.

The compounds of the formula (I) of the invention and their salts, hereinbelow together referred to as compounds of the formula (I) (of the invention), have excellent herbicidal activity against a broad spectrum of economically important monocotyledonous and dicotyledonous weed plants. The active compounds also act efficiently on perennial weeds which produce shoots from rhizomes, rootstocks or other perennial organs and which are difficult to control. In this context, it is generally immaterial whether the substances are applied pre-sowing, pre-emergence or post-emergence.

Specifically, examples may be mentioned of some representatives of the monocotyledonous and dicotyledonous weed flora which can be controlled by the compounds of the invention, without this being intended as a restriction to certain species.

Examples of weed species on which the active compounds act efficiently are, from among the monocotyledons, Agrostis, Alopecurus, Apera, Avena, Brachicaria, Bromus, Dactyloctenium, Digitaria, Echinochloa, Eleocharis, Eleusine, Festuca, Fimbristylis, Ischaemum, Lolium, Monochoria, Panicum, Paspalum, Phalaris, Phleum, Poa, Sagittaria, Scirpus, Setaria, Sphenoclea, and also Cyperus species predominantly from the annual sector, and, from among the perennial species, Agropyron, Cynodon, Imperata and Sorghum, and also perennial Cyperus species.

In the case of dicotyledonous weed species, the spectrum of action extends to species such as, for example, Galium, Viola, Veronica, Lamium, Stellaria, Amaranthus, Sinapis, Ipomoea, Matricaria, Abutilon and Sida from among the annuals, and Convolvulus, Cirsium, Rumex and Artemisia in the case of the perennial weeds. Moreover, herbicidal activity is observed in the case of dicotyledonous weeds such as Ambrosia, Anthemis, Carduus, Centaurea, Chenopodium, Cirsium, Convolvulus, Datura, Emex, Galeopsis, Galinsoga, Lepidium, Lindemia, Papaver, Portlaca, Polygonum, Ranunculus, Rorippa, Rotala, Seneceio, Sesbania, Solanum, Sonchus, Taraxacum, Trifolium, Urtica and Xanthium.

If the compounds of the invention are applied to the soil surface prior to germination, then the weed seedlings are either prevented completely from emerging, or the weeds grow until they have reached the cotyledon stage but then their growth stops, and, eventually, after three to four weeks have elapsed, they die completely.

If the active compounds are applied post-emergence to the green parts of the plants, growth also stops drastically a very short time after the treatment and the weed plants remain at the developmental stage of the point in time of application, or they die completely after a certain time, so that in this manner competition by weeds, which is harmful to the crop plants, is eliminated at a very early point in time and in a sustained manner.

Although the compounds of the invention have an excellent herbicidal activity against monocotyledonous and dicotyledonous weeds, crop plants of economically important crops, for example wheat, barley, rye, rice, corn, sugar beet, cotton and soya, are damaged not at all or only to a negligible extent. For these reasons, the present compounds are highly suitable for selectively controlling unwanted plant growth in plantings of agriculturally useful plants.

In addition, the substances of the invention have outstanding growth regulatory properties in crop plants. They engage in the endogenous plant metabolism with a regulating effect and can thus be employed for the targeted control of plant constituents and for facilitating harvesting, such as, for example, by provoking desiccation and stunted growth. Furthermore, they are also suitable for generally controlling and inhibiting unwanted vegetative growth, without destroying the plants in the process. Inhibition of vegetative growth plays an important role in many monocotyledonous and dicotyledonous crops since it allows lodging to be reduced or prevented completely.

Owing to their herbicidal and plant-growth-regulatory properties, the active compounds can also be employed for controlling weed plants in crops of known or still to be developed genetically engineered plants. The transgenic plants generally have particularly advantageous properties, for example resistance to certain pesticides, in particular certain herbicides, resistance to plant diseases or causative organisms of plant diseases, such as certain insects or microorganisms such as fungi, bacteria or viruses. Other particular properties relate, for example, to the quantity, quality, storage-stability, composition and specific ingredients of the harvested product. Thus, transgenic plants having an increased starch content or a modified quality of the starch or those having a different fatty acid composition of the harvested product are known.

The use of the compounds of the formula (I) of the invention or their salts in economically important transgenic crops of useful and ornamental plants, for example of cereal, such as wheat, barley, rye, oats, millet, rice, manioc and corn, or else in crops of sugar beet, cotton, soya, oilseed rape, potato, tomato, pea and other vegetable species, is preferred.

The compounds of the formula (I) can preferably be used as herbicides in crops of useful plants which are resistant or which have been made resistant by genetic engineering toward the phytotoxic effects of the herbicides.

Conventional ways of preparing novel plants which have modified properties compared to known plants comprise, for example, traditional breeding methods and the generation of mutants. Alternatively, novel plants having modified properties can be generated with the aid of genetic engineering methods (see, for example, EP-A 0221044, EP-A 0131624). For example, there have been descriptions in several cases of
genetically engineered changes in crop plants in order to modify the starch synthesized in the plants (for example WO 92/11376, WO 92/14827 and WO 91/19806),
transgenic crop plants which are resistant to certain herbicides of the glufosinate (cf., for example, EP-A 0242236, EP-A 0242246) or glyphosate type (WO 92/00377), or of the sulfonylurea type (EP-A 0257993, U.S. Pat. No. 5,013,659),
transgenic crop plants, for example cotton, having the ability to produce Bacillus thuringiensis toxins (Bt toxins) which give the plants resistance to certain pests (EP-A 0142924, EP-A 0193259),
transgenic crop plants having a modified fatty acid composition (WO 91/13972).

Numerous molecular-biological techniques which allow the preparation of novel transgenic plants having modified properties are known in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene und Klone” [Genes and Clones], VCH Weinheim, 2nd edition 1996, or Christou, “Trends in Plant Science” 1 (1996) 423-431.

In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments with each other, it is possible to attach adapters or linkers to the fragments.

Plant cells having a reduced activity of a gene product can be prepared, for example, by expressing at least one appropriate antisense-RNA, a sense-RNA to achieve a cosuppression effect, or by expressing at least one appropriately constructed ribozyme which specifically cleaves transcripts of the abovementioned gene product.

To this end, it is possible to employ not only DNA molecules which comprise the entire coding sequence of a gene product, including any flanking sequences that may be present, but also DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical.

When expressing nucleic acid molecules in plants, the synthesized protein can be localized in any desired compartment of the plant cell. However, to achieve localization in a certain compartment, it is, for example, possible to link the coding region with DNA sequences which ensure localization in a certain compartment. Such sequences are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants.

In this manner, it is possible to obtain transgenic plants which have modified properties by overexpression, suppression or inhibition of homologous (=natural) genes or gene sequences or by expression of heterologous (=foreign) genes or gene sequences.

The compounds (I) of the invention can preferably be used in transgenic crops which are resistant to herbicides from the group consisting of the sulfonylureas, glufosinate-ammonium or glyphosate-isopropylammonium and analogous active compounds.

When using the active compounds of the invention in transgenic crops, in addition to the effects against weed plants which can be observed in other crops, there are frequently effects which are specific for the application in the respective transgenic crop, for example a modified or specifically broadened spectrum of weeds which can be controlled, modified application rates which can be used for the application, preferably good combinability with the herbicides to which the transgenic crop is resistant, and an effect on the growth and the yield of the transgenic crop plants.

The invention therefore also provides for the use of the compounds of the formula (I) of the invention as herbicides for controlling weed plants in transgenic crop plants.

The compounds of the invention can be applied in the customary formulations in the form of wettable powders, emulsifiable concentrates, sprayable solutions, dusts or granules. The invention therefore also provides herbicidal and plant growth regulating compositions comprising compounds of the formula (I).

The compounds of the formula (I) can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing compositions, granules for broadcasting and soil application, granules (GR) in the form of microgranules, spray granules, coating granules and adsorption granules, water-dispersible granules (WG), water-soluble granules (SG), ULV formulations, microcapsules, and waxes.

These individual formulation types are known in principle and are described, for example, in Winnacker-Küchler, “Chemische Technologie” [Chemical Technology], Volume 7, C. Hanser Verlag Munich, 4th edition 1986; Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation auxiliaries, such as inert materials, surfactants, solvents and other additives, are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”; 2nd ed., J. Wiley & Sons, N.Y.; C. Marsden, “Solvents Guide”; 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie” [Chemical Technology], Volume 7, C. Hauser Verlag Munich, 4th edition 1986.

Based on these formulations it is also possible to produce combinations with other pesticidally active substances, for example insecticides, acaricides, herbicides and fungicides, and also with safeners, fertilizers and/or growth regulators, for example in the form of a ready-mix formulation or tank mix.

Wettable powders are preparations which are uniformly dispersible in water and which, in addition to the active compound and as well as a diluent or inert substance, also contain surfactants of ionic and/or nonionic type (wetting agents, dispersants), for example polyethoxylated alkyl phenols, polyethoxylated fatty alcohols, polyethoxylated fatty amines, fatty alcohol polyglycol ethersulfates, alkanesulfonates, alkylbenzenesulfonates, sodium ligninsulfonate, sodium 2,2′-dinaphthylmethane-6,6′-disulfonate, sodium dibutylnaphthalenesulfonate or else sodium oleoyl-methyltaurinate. To prepare the wettable powders, the herbicidally active compounds are finely ground, for example in customary apparatuses such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents, with the addition of one or more surfactants of ionic and/or nonionic type (emulsifiers). Examples of emulsifiers which can be used are calcium alkylaryl-sulfonates, such as Ca dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters.

Dusts are obtained by grinding the active compound with finely divided solid substances, for example talc, natural clays, such as kaolin, bentonite and pyrophyllite, or diatomaceous earth.

Suspension concentrates can be water- or oil-based. They can be prepared, for example, by wet milling using commercially customary bead mills, with or without the addition of surfactants, as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers.

Water-dispersible granules are generally prepared by the customary processes, such as spray-drying, fluidized-bed granulation, disk granulation, mixing using high-speed mixers, and extrusion without solid inert material. For the preparation of disk, fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th ed., McGraw-Hill, New York 1973, pp. 8-57.

For further details on the formulation of crop protection products, see for example G. C. Klingman, “Weed Control as a Science”, John Wiley and Sons., Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical formulations generally contain from 0.1 to 99% by weight, in particular from 0.1 to 95% by weight, of active compound of the formula (I). In wettable powders the concentration of active compound is, for example, from about 10 to 90% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules, the content of active compound depends partly on whether the active compound is in liquid or solid form and on the granulation auxiliaries, fillers, etc. that are used. In water-dispersible granules the content of active compound, for example, is between 1 and 95% by weight, preferably between 10 and 80% by weight.

In addition, said formulations of active compound may comprise the stickers, wetters, dispersants, emulsifiers, penetrants, preservatives, antifreeze agents, solvents, fillers, carriers, colorants, antifoams, evaporation inhibitors and pH and viscosity regulators which are customary in each case.

The compounds of the formula (I) or their salts can be used as such or combined in the form of their preparations (formulations) with other pesticidally active substances, such as, for example, insecticides, acaricides, nematicides, herbicides, fungicides, safeners, fertilizers and/or growth regulators, for example as ready-mix formulations or tank mixes. Suitable active compounds which can be combined with the active compounds of the invention in mixed formulations or in a tank mix are, for example, known active compounds, whose effect is based on an inhibition of, for example, acetolactate synthase, acetyl-CoA carboxylase, PS I, PS II, HPPDO, phytoene desaturase, protoporphyrinogen oxidase, glutamine synthetase, cellulose biosynthesis, 5-enolpyruvylshikimate-3-phosphate synthetase. Such compounds, and also other compounds that can be used, with a mechanism of action that is, in some cases, unknown or different, are described, for example, in Weed Research 26, 441-445 (1986), or in “The Pesticide Manual”, 11^{th} edition 1997 (hereafter also abbreviated to “PM”) and 12^{th} edition 2000, The British Crop Protection Council and the Royal Soc. of Chemistry (publisher), and in the literature cited therein. For example, the following active compounds may be mentioned as herbicides which are known from the literature and which can be combined with the compounds of the formula (I) (note: the compounds are either referred to by the “common name” in accordance with the International Organization for Standardization (ISO) or by the chemical names, if appropriate together with a customary code number): acetochlor; acifluorfen(-sodium); aclonifen; AKH 7088, i.e. [[[1-[5-[2-chloro-4-(trifluoromethyl)phenoxy]-2-nitrophenyl]-2-methoxyethylidene]amino]oxy]acetic acid and its methyl ester; alachlor; alloxydim(-sodium); ametryn; amicarbazone; amidochlor, amidosulfuron; amitrol; AMS, i.e. ammonium sulfamate; anilofos; asulam; atrazine; azafenidin, azimsulfuron (DPX-A8947); aziprotryn; barban; BAS 516H, i.e. 5-fluoro-2-phenyl-4H-3,1-benzoxazin-4-one; beflubutamide; benazolin(-ethyl); benfluralin; benfuresate; bensulfuron(-methyl); bensulide; bentazone; benzobicyclone, benzofenap; benzofluor; benzoylprop(-ethyl); benzthiazuron; bialaphos; bifenox; bispyribac(-sodium); bromacil; bromobutide; bromofenoxim; bromoxynil; bromuron; buminafos; busoxinone; butachlor; butafenacil, butamifos; butenachlor; buthidazole; butralin; butroxydim, butylate; cafenstrole (CH-900); carbetamide; cafentrazone(-ethyl); caloxydim, CDAA, i.e. 2-chloro-N,N-di-2-propenylacetamide; CDEC, i.e. 2-chloroallyl diethyldithio-carbamate; chlomethoxyfen; chloramben; chlorazifop-butyl, chlormesulon; chlorbromuron; chlorbufam; chlorfenac; chlorflurecol-methyl; chloridazon; chlorimuron(-ethyl); chlornitrofen; chlorotoluron; chloroxuron; chlorpropham; chlorsulfuron; chlorthal-dimethyl; chlorthiamid; chlortoluron, cinidon(-ethyl and -methyl); cinmethylin; cinosulfuron; clefoxydim, clethodim; clodinafop and its ester derivatives (for example clodinafop-propargyl); clomazone; clomeprop; cloproxydim; clopyralid; clopyrasulfuron(-methyl), cloransulam(-methyl), cumyluron (JC 940); cyanazine; cycloate; cyclosulfamuron (AC 104); cycloxydim; cycluron; cyhalofop and its ester derivatives (for example butyl ester, DEH-112); cyperquat; cyprazine; cyprazole; daimuron; 2,4-D; 2,4-DB; dalapon; desmedipham; desmetryn; di-allate; dicamba; dichlobenil; dichlorprop; diclofop and its esters such as diclofop-methyl; diclosulam, diethatyl(-ethyl); difenoxuron; difenzoquat; diflufenican; diflufenzopyr, dimefuron; dimepiperate, dimethachlor; dimethametryn; dimethenamid (SAN-582H); dimethazone, dimexyflam, dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole-4-carboxamide; endothal; epoprodan, EPTC; esprocarb; ethalfluralin; ethametsulfuron-methyl; ethidimuron; ethiozin; ethofumesate; ethoxyfen and its esters (for example ethyl ester, HN-252); ethoxysulfuron, etobenzanid (HW 52); F5231, i.e. N-[2-chloro-4-fluoro-5-[4-(3-fluoropropyl)-4,5-dihydro-5-oxo-1H-tetrazol-1-yl]-phenyl]ethanesulfonamide; fenoprop; fenoxan, fenoxaprop and fenoxaprop-P and their esters, for example fenoxaprop-P-ethyl and fenoxaprop-ethyl; fenoxydim; fentrazamide; fenuron; flamprop(-methyl or -isopropyl or -isopropyl-L); flazasulfuron; floazulate, florasulam, fluazifop and fluazifop-P and their esters, for example fluazifop-butyl and fluazifop-P-butyl; flucarbazone(-sodium), fluchloralin; flumetsulam; flumeturon; flumiclorac(-pentyl), flumioxazin (S-482); flumipropyn; fluometuron, fluorochloridone, fluorodifen; fluoroglycofen(-ethyl); flupoxam (KNW-739); flupropacil (UBIC-4243); flupyrsulfuron(-methyl, or -sodium), flurenol(-butyl), fluridone; flurochloridone; fluroxypyr(-meptyl); flurprimidol, flurtamone; fluthiacet-(-methyl), fluthiamide, fomesafen; foramsulfuron, fosamine; furyloxyfen; glufosinate(-ammonium); glyphosate(-isopropylammonium); halosafen; halosulfuron(-methyl) and its esters (for example methyl ester, NC-319); haloxyfop and its esters; haloxyfop-P (=R-haloxyfop) and its esters; hexazinone; imazamethabenz(-methyl); imazapyr; imazaquin and salts such as the ammonium salt; imazamethapyr, imazamox, imazapic, imazethamethapyr; imazethapyr, imazosulfuron; indanofan, ioxynil; isocarbamid; isopropalin; isoproturon; isouron; isoxaben; isoxachlortole, isoxaflutole, isoxapyrifop; karbutilate; lactofen; lenacil; linuron; MCPA; MCPB; mecoprop; mefenacet; mefluidid; mesosulfuron, mesotrione, metamitron; metazachlor; methabenzthiazuron; metham; methazole; methoxyphenone; methyldymron; metabenzuron, methobenzuron; metobromuron; (alpha-)metolachlor; metosulam (XRD 511); metoxuron; metribuzin; metsulfuron-methyl; MH; molinate; monalide; monocarbamide dihydrogensulfate; monolinuron; monuron; MT 128, i.e. 6-chloro-N-(3-chloro-2-propenyl)-5-methyl-N-phenyl-3-pyridazinamine; MT 5950, i.e. N-[3-chloro-4-(1-methylethyl)-phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazone; oxasulfuron; oxaciclomefone; oxyfluorfen; paraquat; pebulate; pelargonic acid, pendimethalin; pentoxazone, perfluidone; phenisopham; phenmedipham; picloram; picolinafen, pinoxaden, piperophos; piributicarb; pirifenop-butyl; pretilachlor; primisulfuron-(-methyl); procarbazone(-sodium), procyazine; prodiamine; profluralin; proglinazine-(-ethyl); prometon; prometryn; propachlor; propanil; propaquizafop and its esters; propazine; propham; propisochlor; propyzamide; prosulfalin; prosulfocarb; prosulfuron (CGA-152005); prynachlor; pyraflufen(-ethyl), pyrazolinate; pyrazon; pyrazosulfuron(-ethyl); pyrazoxyfen; pyribenzoxim, pyributicarb, pyridafol, pyridate; pyriminobac(-methyl), pyrithiobac(-sodium) (KIH-2031); pyroxofop and its esters (for example propargyl ester); quinclorac; quinmerac; quinoclamine, quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetra-hydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and methyl ester; sulcotrione, sulfentrazone (FMC-97285, F-6285); sulfazurone; sulfometuron(-methyl); sulfosate (ICI-A0224); sulfosulfuron, TCA; tebutam (GCP-5544); tebuthiuron; tepraloxydim, terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiafluamide; thiazafluron; thiazopyr (Mon-13200); thidiazimin (SN-24085); thifensulfuron(-methyl); thiobencarb; tiocarbazil; tralkoxydim; tri-allate; triasulfuron; triaziflam, triazofenamide; tribenuron(-methyl); triclopyr; tridiphane; trietazine; trifluralin; triflusulfuron and esters (e.g. methyl ester, DPX-66037); trimeturon; tritosulfuron, tsitodef; vernolate; WL 110547, i.e. 5-phenoxy-1-[3-(trifluoromethyl)phenyl]-1H-tetrazole; BAY MKH 6561, UBH-509; D489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127, KIH-485, and KIH-2023.

Of particular interest is the selective control of weed plants in crops of useful and ornamental plants. Although the compounds (I) of the invention have very good to satisfactory selectivity in a large number of crops, it is possible in principle that phytotoxicity in the crop plants can occur in some crops and, in particular, when the compounds (I) are mixed with other herbicides which are less selective. In this respect, combinations of the compounds of the formula (I) of the invention which contain the compounds of the formula (I), or their combinations with other herbicides or pesticides, and safeners are of particular interest. The safeners, which are employed in such amounts that they act as antidotes, reduce the phytotoxic side effects of the herbicides/pesticides used, for example in economically important crops such as cereals (wheat, barley, rye, corn, rice, millet), sugar beet, sugar cane, oilseed rape, cotton and soya, preferably cereal. Suitable safeners for the compounds (I) and their combinations with other pesticides are, for example, the following groups of compounds:
a) Compounds of the type of dichlorophenylpyrazoline-3-carboxylic acid, preferably compounds such as ethyl 1-(2,4-dichlorophenyl)-5-(ethoxycarbonyl)-5-methyl-2-pyrazoline-3-carboxylate (S1-1) (“mefenpyr-diethyl”, PM, pp. 781-782), and related compounds, as described in WO 91/07874,
b) derivatives of dichlorophenylpyrazole carboxylic acid, preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S14), ethyl 1-(2,4-dichlorophenyl)-5-phenylpyrazole-3-carboxylate (S1-5) and related compounds, as described in EP-A-333 131 and EP-A-269 806.
c) Compounds of the type of the triazolecarboxylic acids, preferably compounds such as fenchlorazole(ethyl ester), i.e. ethyl 1-(2,4-dichlorophenyl)-5-trichloromethyl-(1H)-1,2,4-triazole-3-carboxylate (S1-6) and related compounds as described in EP-A-174 562 and EP-A-346 620.
d) Compounds of the type of the 5-benzyl- or 5-phenyl-2-isoxazoline-3-carboxylic acid, or the 5,5-diphenyl-2-isoxazoline-3-carboxylic acid, preferably compounds such as ethyl 5-(2,4-dichlorobenzyl)-2-isoxazoline-3-carboxylate (S1-7) or ethyl 5-phenyl-2-isoxazoline-3-carboxylate (S1-8) and related compounds, as described in WO 91/08202, or ethyl 5,5-diphenyl-2-isoxazolinecarboxylate (S1-9) (“isoxadifen-ethyl”) or its -n-propyl ester (S1-10) or ethyl 5-(4-fluorophenyl)-5-phenyl-2-isoxazoline-3-carboxylate (S1-11), as described in the German patent application (WO-A-95/07897).
e) Compounds of the type of the 8-quinolineoxyacetic acid (S2), preferably 1-methylhex-1-yl (5-chloro-8-quinolineoxy)acetate (common name “cloquintocet-mexyl” (S2-1) (see PM, pp. 263-264) 1,3-dimethylbut-1-yl (5-chloro-8-quinolineoxy)acetate (S2-2), 4-allyloxybutyl (5-chloro-8-quinolineoxy)acetate (S2-3), 1-allyloxyprop-2-yl (5-chloro-8-quinolineoxy)acetate (S24), ethyl (5-chloro-8-quinolineoxy)acetate (S2-5), methyl (5-chloro-8-quinolineoxy)acetate (S2-6), allyl (5-chloro-8-quinolineoxy)acetate (S2-7), 2-(2-propylideneiminoxy)-1-ethyl (5-chloro-8-quinolineoxy)acetate (S2-8), 2-oxoprop-1-yl (5-chloro-8-quinolineoxy)acetate (S2-9) and related compounds, as described in EP-A-86 750, EP-A-94 349 and EP-A-191 736 or EP-A-0 492 366.
f) Compounds of the type of the (5-chloro-8-quinolineoxy)malonic acid, preferably compounds such as diethyl (5-chloro-8-quinolineoxy)malonate, diallyl (5-chloro-8-quinolineoxy)malonate, methyl ethyl (5-chloro-8-quinolineoxy)malonate and related compounds, as described in EP-A-0 582 198.
g) Active compounds of the type of the phenoxyacetic or -propionic acid derivatives or the aromatic carboxylic acids, such as, for example, 2,4-dichlorophenoxyacetic acid (esters) (2,4-D), 4-chloro-2-methylphenoxypropionic esters (Mecoprop), MCPA or 3,6-dichloro-2-methoxybenzoic acid (esters) (Dicamba).
h) Active compounds of the type of the pyrimidines, which are used as soil-acting safeners in rice, such as, for example,
   “fenclorim” (PM, pp. 512-511) (=4,6-dichloro-2-phenylpyrimidine), which is known as safener for pretilachlor in sown rice,
i) active compounds of the type of the dichloroacetamides, which are frequently used as pre-emergent safeners (soil-acting safeners), such as, for example,
   “dichlormid” (PM, pp. 363-364) (=N,N-diallyl-2,2-dichloroacetamide),
   “R-29148” (=3-dichloroacetyl-2,2,5-trimethyl-1,3-oxazolidine from Stauffer),
   “benoxacor” (PM, pp. 102-103) (=4-dichloroacetyl-3,4-dihydro-3-methyl-2H-1,4-benzoxazine),
   “PPG-1292” (=N-allyl-N-[(1,3-dioxolan-2-yl)methyl]dichloroacetamide from PPG Industries),
   “DK-24” (=N-allyl-N-[(allylaminocarbonyl)methyl]dichloroacetamide from Sagro-Chem),
   “AD-67” or “MON 4660” (=3-dichloroacetyl-1-oxa-3-aza-spiro[4,5]decane from Nitrokemia or Monsanto),
   “diclonon” or “BAS1 45138” or “LAB145138” (=3-dichloroacetyl-2,5,5-trimethyl-1,3-diazabicyclo[4.3.0]nonane from BASF) and
   “furilazol” or “MON 13900” (see PM, 637-638) (=(RS)-3-dichloroacetyl-5-(2-furyl)-2,2-dimethyloxazolidine)
j) Active compounds of the type of the dichloroacetone derivatives, such as, for example,
   “MG 191” (CAS-Reg. No. 96420-72-3) (=2-dichloromethyl-2-methyl-1,3-dioxolane from Nitrokemia), which is known as safener for corn,
k) active compounds of the type of the oxyimino compounds, which are known as seed dressings, such as, for example,
   “oxabetrinil” (PM, pp. 902-903) (=(Z)-1,3-dioxolan-2-ylmethoxyimino-(phenyl)acetonitrile), which is known as seed dressing safener for millet against metolachlor damage,
   “fluxofenim” (PM, pp. 613-614) (=1-(4-chlorophenyl)-2,2,2-trifluoro-1-ethanone O-(1,3-dioxolan-2-ylmethyl) oxime), which is known as seed dressing safener for millet against metolachlor damage,
   “cyometrinil” or “-CGA-43089” (PM, p. 1304) (=(Z)-cyanomethoxyimino-(phenyl)acetonitrile), which is known as seed dressing safener for millet against metolachlor damage,
l) active compounds of the type of the thiazolecarboxylic esters, which are known as seed dressings, such as, for example,
   “flurazol” (PM, pp. 590-591) (=benzyl 2-chloro-4-trifluoromethyl-1,3-thiazole-5-carboxylate), which is known as seed dressing safener for millet against alachlor and metolachlor damage,
m) active compounds of the type of the naphthalenedicarboxylic acid derivatives, which are known as seed dressings, such as, for example,
   “naphthalic anhydride” (PM, p. 1342) (=1,8-naphthalenedicarboxylic anhydride), which is known as seed dressing safener for corn against thiocarbamate herbicide damage,
n) active compounds of the type of the chromanacetic acid derivatives, such as, for example,
   “CL 304415” (CAS-Reg. No. 31541-57-8) (=2-(4-carboxychroman-4-yl)acetic acid from American Cyanamid), which is known as safener for corn against imidazolinone damage,
o) active compounds which, in addition to a herbicidal action against weed plants, also have safener action in crop plants such as rice, such as, for example,
   “dimepiperate” or “MY-93” (PM, pp. 404-405) (=S-1-methyl-1-phenylethyl piperidine-1-thiocarboxylate), which is known as safener for rice against herbicide molinate damage,
   “daimuron” or “SK 23” (PM, p. 330) (=1-(1-methyl-1-phenylethyl)-3-p-tolyl-urea), which is known as safener for rice against herbicide imazosulfuron damage,
   “cumyluron”=“JC-940” (=3-(2-chlorophenylmethyl)-1-(1-methyl-1-phenylethyl)urea, see JP-A-60087254), which is known as safener for rice against damage by some herbicides,
   “methoxyphenon” or “NK 049” (=3,3′-dimethyl-4-methoxy-benzophenone), which is known as safener for rice against damage by some herbicides,
   “CSB” (=1-bromo-4-(chloromethylsulfonyl)benzene) (CAS-Reg. No. 54091-064 from Kumiai), which is known as safener against damage by some herbicides in rice
p) N-Acylsulfonamides of the formula (S3) and salts thereof, 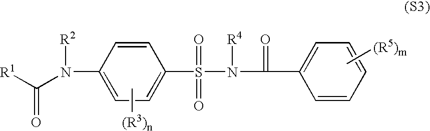
   as described in WO-A-97/45016,
q) acylsulfamoylbenzoamides of the formula (S4), if appropriate also in salt form, 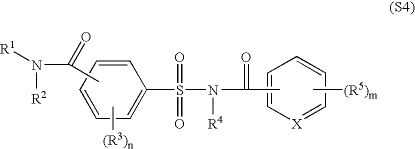
   as described in the International Application No. PCT/EP98/06097, and
r) compounds of the formula (S5), 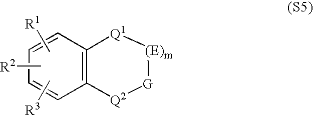
   as described in WO-A98/13 361,
   including the stereoisomers and the salts used in agriculture.

Among the safeners mentioned, (S1-1) and (S1-9) and (S2-1), in particular (S1-1) and (S1-9), are of particular interest.

Some of the safeners are already known as herbicides and consequently show not only the herbicidal action against weed plants but also protective action in the crop plants.

The ratios by weight of herbicide (mixture) to safener generally depend on the application rate of the herbicide and the efficacy of the safener in question and can vary within wide limits, for example in the range from 200:1 to 1:200, preferably 100:1 to 1:100, in particular 20:1 to 1:20. Analogously to the compounds of the formula (I) or their mixtures, the safeners can be formulated with other herbicides/pesticides and can be provided and used as ready-mix formulations or tank mixes with the herbicides.

For use, the formulations which are present in commercially available form are, if appropriate, diluted in the customary manner, for example using water in the case of wettable powders, emulsifiable concentrates, dispersions and water-dispersible granules. Preparations in the form of dusts, granules for soil application or broadcasting and sprayable solutions are usually not further diluted with other inert substances prior to use.

The application rate of the compounds of the formula (I) required varies with the external conditions, such as temperature, humidity, the nature of the herbicide used and the like. It can vary within wide limits, for example between 0.001 and 10.0 kg/ha or more of active substance, but it is preferably between 0.005 and 5 kg/ha.

### A. SYNTHESIS EXAMPLES

In the text below, a number of examples of the synthesis of compounds of the formula (I) or salts thereof are described by way of example.

### Synthesis Example 1

### 3-{[(Chloro(phenyl)methyl]sulfinyl}-5,5-dimethyl-4,5-dihydroisoxazole

4.879 g (41 mmol) of sodium dihydrogen phosphate were dissolved in 10 ml of water and this solution was admixed with 2.00 g (9 mmol) of 3-benzylthio-5,5-dimethyl-4,5-dihydroisoxazole in solution in 50 ml of dichloromethane. Chlorine gas was introduced at 0-5° C. for 10 minutes followed by stirring at 0-5° C. for 30 minutes. Thereafter there followed introduction of chlorine gas again at 0-5° C. for 10 minutes and subsequent stirring at 0-5° C. for 15 minutes until TLC monitoring indicated that all of the starting material had undergone reaction. The phases were separated and extraction was carried out three times with ethyl acetate. The combined phases were washed with water and then dried over magnesium sulfate, filtered, and concentrated. Column chromatography of the crude product gave 444 mg of a diastereomer mixture of 3-{[(chloro(phenyl)methyl]sulfinyl}-5,5-dimethyl-4,5-dihydro-isoxazole in the form of colorless crystals.

¹H NMR (CDCl₃) diastereomer mixture: 5.58 (s, 1H, (SO)CHCl) and 5.76 (s, 1H, (SO)CHCl)

### Synthesis Example 2

### 3-{[Chloro(phenyl)methyl]sulfonyl}-5,5-dimethyl-4,5-dihydroisoxazole

A solution of 150 mg of 3-{[chloro(phenyl)methyl]sulfinyl}-5,5-dimethyl-4,5-dihydro-isoxazole (synthesis example 1) in 10 ml of methylene chloride was admixed at 0-5° C. with 1.1 equivalents of m-chloroperbenzoic acid and the mixture was subsequently stirred at 0-5° C. for 30 minutes. It was allowed to return to room temperature and then stirred for a further 12 hours. 2 N sodium hydroxide solution was added to the reaction solution, the solution was stirred for a few minutes subsequently, and the organic phase was separated off. The aqueous phase was extracted twice with dichloromethane. The combined phases were washed with water and then dried over magnesium sulfate, filtered, and concentrated. Column chromatography of the crude product gave 115 mg of 3-(chlorophenyl-methanesulfonyl)-5,5-dimethyl-4,5-dihydroisoxazole in the form of a white solid.

¹H NMR (CDCl₃) 6.00 (s, 1H, (SO₂)CHCl)

### Synthesis Example 3

### 3-{[Chloro(2-chloro-6-fluorophenyl)methyl]sulfonyl}-5,5-dimethyl-4,5-dihydro-isoxazole

200 mg of 3-[(2-chloro-6-fluorobenzyl)sulfonyl]-5,5-dimethyl-4,5-dihydroisoxazole (WO 2001 012613) were added at 0° C. to a solution of 1.25 equivalents of pulverized sodium hydroxide and 1.1 equivalents of tetrachloromethane in 5 ml of dimethylformamide at 0-5° C. and the mixture was subsequently stirred at 0-5° C. for 1 hour. Over the course of 12 hours it was allowed to return to room temperature, water was added to the reaction solution, which was then stirred subsequently for a few minutes and extracted three times with ethyl acetate. The combined phases were washed with water and then dried over magnesium sulfate, filtered, and concentrated. Column chromatography of the crude product gave 110 mg of 3-{[chloro(2-chloro-6-fluorophenyl)methyl]sulfonyl}-5,5-dimethyl-4,5-dihydroisoxazole in the form of a yellowish solid.

¹H NMR (CDCl₃) 6.79 (d, 1H, (SO₂)CHCl)

### Synthesis Example 4

### 3-{[Chloro(2-chloro-6-fluorophenyl)methyl]sulfinyl}-5,5-dimethyl-4,5-dihydroisoxazole

434 mg (4 mmol) of sodium dihydrogen phosphate were dissolved in 1 ml of water and this solution was admixed with 220 mg (1 mmol) of 3-[(2-chloro-6-fluoro-benzyl)thio]-5,5-dimethyl-4,5-dihydroisoxazole in solution in 5 ml of dichloromethane. Chlorine gas was introduced at 0-5° C. for 10 minutes, followed by stirring at 0-5° C. for 30 minutes. The phases were separated and the organic phase was washed three times with ice-water, then dried over magnesium sulfate, filtered, and concentrated. Column chromatography of the crude product gave 60 mg of a diastereomer mixture of 3-{[Chloro(2-chloro-6-fluorophenyl)methyl]sulfinyl}-5,5-dimethyl-4,5-dihydro-isoxazole in the form of a yellowish semicrystalline mass.

¹H NMR (CDCl₃) diastereomer mixture: 6.20 (d, 1H, (SO)CHCl) and 6.22 (s, 1H, (SO)CHCl).

### Synthesis Example 5

### 3-{[Dichloro(3-fluorophenyl)methyl]sulfonyl}-5-ethyl-5-methyl-4,5-dihydroisoxazole

A suspension of 136 mg (3 mmol) of finely pulverized caustic soda in 10 ml of dimethylformamide was admixed with 1.046 g (7 mmol) of carbon tetrachloride and then with 970 mg (3 mmol) of 5-ethyl-3-[(3-fluorophenyl)methylsulfonyl]-5-methyl-4,5-dihydroisoxazole (WO 2001 012613). The mixture was stirred vigorously at room temperature for 2 hours. After dilution with water it was extracted with dichloromethane, and the organic phase was dried and concentrated. Purification was carried out by chromatography on silica gel. This gave 285 mg (22.5% of theory) of product as a pale yellow solid of m.p. 100° C.

### Synthesis Example 6

### 3-({Bromo[5-(difluoromethoxy)-1-methyl-3-(trifluoromethyl)-1H-pyrazol-4-yl]-methyl}sulfonyl)-5,5-dimethyl-4,5-dihydroisoxazole

A forcefully stirred mixture of 0.308 g (1 mmol) of carbon tetrabromide and 0.042 g (1 mmol) of finely pulverized caustic soda in 20 ml of dimethylformamide was admixed at 0° C. with 0.330 g (1 mmol) of 3-({[5-(difluoromethoxy)-1-methyl-3-(trifluoromethyl)-1H-pyrazol-4-yl]methyl}sulfonyl)-5,5-dimethyl-4,5-dihydroisoxazole (WO 2004 014138) and the mixture was stirred at 0° C. for an hour. After overnight standing it was diluted with water and taken up in ethyl acetate and the organic phase was dried and concentrated. Purification was carried out by chromatography on silica gel (heptane/ethyl acetate 5:1). This gave 0.230 g of product as a colorless oil (55.1% of theory)

NMR (CDCl₃, 300 MHz): 1.55 (s, 6H, 2CH₃); 3.15 (AB, 2H, isoxazoline-CH₂); 3.90 (s, 3H, NCH₃); 6.01 (s, 1H, CHBr); 7.00 (dd, 1H, OCF₂H)

### Synthesis Example 7

### 3-{[(2,5-Difluorophenyl)(fluoro)methyl]sulfonyl}-5,5-dimethyl-4,5-dihydroisoxazole

A solution of 370 mg (1.3 mmol) of 3-(2,5-difluorobenzylsulfonyl)-5,5-dimethyl-4,5-dihydroisoxazole (WO 2001 012613) in 10 ml of dimethylformamide was admixed with 1.5 ml of a 1M solution of potassium tert-butoxide in tetrahydrofuran. The mixture was stirred at room temperature for 10 minutes, then admixed with 565 mg (1.8 mmol) of N-fluorobenzenesulfonimide (AccuFluor™), and stirred again at room temperature for 8 hours. The reaction mixture was diluted with saturated sodium bicarbonate solution and extracted by shaking with dichloromethane. The organic phase was twice extracted by shaking with water, dried, and concentrated. Purification was carried out by chromatography on silica gel (heptane/ethyl acetate 5:1). This gave 296 mg of product (74.2% of theory) as a colorless oil.

NMR (300 MHz, CDCl3): 1.55 (2s, 6H, 2 CH₃); 3.15 (AB, 2H, isoxazoline-CH₂); 6.74 (d, 1H, CHF), 7.20 (m, 2H, phenyl-H); 7.38 (m, 1H, phenyl-H)

### Synthesis Example 8

### 3-{[(2,6-Difluorophenyl)(fluoro)methyl]sulfinyl}-5,5-dimethyl-4,5-dihydroisoxazole

2.90 g (8 mmol) of 1-chloromethyl-4-fluoro-1,4-diazabicyclo[2.2.2]octane bistetrafluoroborate (F-TEDA-BF₄, Selectfluor™) in 40 ml of acetonitrile were introduced as an initial charge. Subsequently a solution of 2.00 g (8 mmol) of 3-(2,6-difluorobenzylthio)-5,5-dimethyl-4,5-dihydroisoxazole (WO 2001 012613) in a little acetonitrile was added dropwise at room temperature. After 10 minutes 0.98 g (10 mmol) of triethylamine was added. After four hours of stirring at room temperature the batch was poured into water and extracted by stirring with dichloromethane. The organic phase was dried and concentrated. The residue was dissolved in 50 ml of dichloromethane, and 25 ml of this solution was admixed in portions with one equivalent of 3-chloroperbenzoic acid, with ice-bath cooling, and subsequently stirred at room temperature for 6 hours. It was then extracted twice by stirring with dilute aqueous sodium hydroxide solution, and the organic phase was dried and concentrated. Purification was carried out by chromatography on silica gel (heptane/ethyl acetate 4:1). This gave 0.27 g of product as a colorless solid of m.p. 87° C. The product consists of a diastereomer mixture in a ratio of approximately 9:1.

NMR (CDCl₃, 300 MHz)

Principal isomer: 1.51, 1.55 (2s, 6H, 2CH₃); 3.13 (AB, 2H, isoxazoline-CH₂); 6.57 (d, 1H, CHF), 7.02, (m, 2H, phenyl-H); 7.50, (m, 1H, phenyl-H)

Secondary isomer: 1.36, 1.45 (2s, 6H, 2CH₃); 3.09 (AB, 2H, isoxazoline-CH₂); 6.52 (d, 1H, CHF), 7.02, (m, 2H, phenyl-H); 7.50, (m, 1H, phenyl-H)

### Synthesis Example 9

### 3-{[(2,6-Difluorophenyl)(fluoro)methyl]sulfonyl}-5,5-dimethyl-4,5-dihydroisoxazole

For this synthesis example the alpha-fluorothioether was first prepared as in synthesis example 9. For the reaction to the alpha-fluorosulfone, the 25 ml of the solution of the fluorothioether in dichloromethane left over from example 9 were used. This solution was admixed with one equivalent of 3-chloroperbenzoic acid in portions with ice-bath cooling. The subsequent procedure was entirely analogous to that of example 9. This gave 0.18 g of product as a colorless solid of m.p. 88° C. By-products were starting material and the sulfoxide described in example 8.

NMR (CDCl₃, 300 MHz): 1.55 (s, 6H, 2CH₃); 3.19 (AB, 2H, isoxazoline-CH₂); 6.72 (d, 1H, CHF), 7.03, (tr, 2H, phenyl-H); 7.51, (m, 1H, phenyl-H)

### Synthesis Example 10

### 3-{[Dibromo(phenyl)methyl]sulfonyl}-5,5-dimethyl-4,5-dihydroisoxazole and 3-{[bromo(phenyl)methyl]sulfonyl}-5,5-dimethyl-4,5-dihydroisoxazole

A forcefully stirred mixture of 0.576 g of carbon tetrabromide and 0.079 g of finely pulverized caustic soda in 15 ml of dimethylformamide was admixed at 0° C. with 400 mg of 3-[benzylsulfonyl]-5,5-dimethyl-4,5-dihydroisoxazole (WO 2001 012613) and the mixture was subsequently stirred at 0° C. for an hour. After overnight standing it was diluted with water and taken up in ethyl acetate and the organic phase was dried and concentrated. Purification was carried out by chromatography on silica gel. This gave 20 mg (2.9% of theory) of 3-{[dibromo(phenyl)methyl]sulfonyl}-5,5-dimethyl-4,5-dihydroisoxazole as a yellow solid and 400 mg (72% of theory) of 3-{[bromo(phenyl)methyl]sulfonyl}-5,5-dimethyl-4,5-dihydroisoxazole as a white solid.

### 3-{[Bromo(phenyl)methyl]sulfonyl}-5,5-dimethyl-4,5-dihydroisoxazole

¹H NMR (CDCl₃): 6.03 (s, 1H, (SO₂)CHBr)

### 3-{[Dibromo(phenyl)methyl]sulfonyl}-5,5-dimethyl-4,5-dihydroisoxazole

¹H NMR (CDCl₃): 2.77 (s, 2H, CH₂)

### Synthesis Example 11

### 3-{[Difluoro(phenyl)methyl]sulfonyl}-5-ethyl-5-methyl-4,5-dihydroisoxazole and 5-ethyl-3-{[fluoro(phenyl)methyl]sulfonyl}-5-methyl-4,5-dihydroisoxazole

In the same way as in synthesis example 7, 1.420 g (5 mmol) of 3-(benzylsulfonyl)-5-ethyl-5-methyl-4,5-dihydroisoxazole, 2.345 g (7 mmol) of N-fluorodibenzenesulfonimide (AccuFluor™) and 6.4 ml of a 1M solution of potassium tert-butoxide in tetrahydrofuran were reacted. Purification was carried out by chromatography on silica gel (heptane/ethyl acetate 9:1). Eluted first from the column were 172 mg (10.1% of theory) of 3-{[difluoro(phenyl)methyl]sulfonyl}-5-ethyl-5-methyl-4,5-dihydroisoxazole as a colorless solid (m.p. 61° C.). As a second fraction, 5-ethyl-3-{[fluoro(phenyl)methyl]sulfonyl}-5-methyl-4,5-dihydroisoxazole was obtained as a diastereomer mixture (ratio approximately 1:1).

### 3-{[Difluoro(phenyl)methyl]sulfonyl}-5-ethyl-5-methyl-4,5-dihydroisoxazole

NMR (400 MHz, CDCl3): 1.00 (tr, 3H, CH₃CH₂); 1.50 (s, 3H, CH₃); 1.82 (q, 2H, CH₃CH₂); 3.12 (AB, 2H, isoxazoline-CH₂); 7.55 (tr, 2H, phenyl-H); 7.67 (tr, 1H, phenyl-H); 7.72 (d, 2H, phenyl-H)

### 5-Ethyl-3-{[fluoro(phenyl)methyl]sulfonyl}-5-methyl-4,5-dihydroisoxazole

(Diastereomer mixture approximately 1:1)

NMR (400 MHz, CDCl3): 0.97 (tr, 3H, CH₃CH₂); 1.46 (s, 3H, CH₃); 1.78 (q, 2H, CH₃CH₂); 3.03, 3.06 (2AB, 2H, isoxazoline-CH₂); 6.38 (d, 1H, CHF); 7.45-7.62 (m, 5H, phenyl-H)

The compounds described in table A below are obtained in accordance with or in analogy to the synthesis examples 1 to 11 described above.

Key:

Me=methyl

Et=ethyl

Ph=phenyl

Pr=propyl

i-Pr=isopropyl

LENGTHY TABLE REFERENCED HERE
US20070161513A1-20070712-T00001
Please refer to the end of the specification for access instructions.

Set out below by way of example are the physical parameters of certain of the compounds of the invention identified in table A.

All NMR measurements were carried out in CDCl₃ using a 300 MHz instrument from Varian.

Compound I-1.1

m.p.: 110° C.

Compound I-2.2 (diastereomer mixture approximately: 1:1)

0.97, 1.01 (2tr, 3H, CH₃CH₂); 1.48, 1.52 (2s, 3H, CH₃); 1.88 (m, 2H, CH₃CH₂); 3.09 (2AB, 2H, isoxazoline-CH₂); 6.37 (s, 1H, CHCl); 7.15 (m, 2H, phenyl-H); 7.50 (s, 1H, phenyl-H)

Compound I-3.2 (diastereomer mixture approximately: 1:1)

0.98, 1.00 (2tr, 3H, CH₃CH₂); 1.50 (2s, 3H, CH₃); 1.80 (2m, 2H, CH₃CH₂); 3.16 (2AB, 2H, isoxazoline-CH₂); 6.42 (s, 1H, CHCl); 7.02 (tr, 2H, phenyl-H); 7.49 (m, 1H, phenyl-H);

m.p.: 120° C.

Compound I-4.2 (diastereomer mixture approximately: 1:1)

0.99, 1.03 (2tr, 3H, CH₃CH₂); 1.48, 1.52 (2s, 3H, CH₃); 1.80 (m, 2H, CH₃C₂); 3.10, 3.18 (2AB, 2H, isoxazoline-CH₂); 6.42 (s, 1H, CHCl); 7.31 (tr, 1H, phenyl-H); 7.76 (m, 1H, phenyl-H); 8.08 (dd, 1H, phenyl-H)

Compound I-6.2 (diastereomer mixture approximately: 1:1)

0.91, 0.98 (2tr, 3H, CH₃CH₂); 1.40, 1.49 (2s, 3H, CH₃); 1.76 (m, 2H, CH₃CH₂); 2.97, 3.01 (2AB, 2H, isoxazoline-CH₂); 6.69 (2s, 1H, CHCl); 7.45 (m, 3H, phenyl-H); 7.90 (m, 1H, phenyl-H)

Compound I-7.2 (diastereomer mixture approximately: 1:1)

0.93, 1.00 (2tr, 3H, CH₃CH₂); 1.41, 1.48 (2s, 3H, CH₃); 1.78 (m, 2H, CH₃CH₂); 3.05 (2AB, 2H, isoxazoline-CH₂); 6.41 (2s, 1H, CHCl); 7.15 (tr, 1H, phenyl-H); 7.29 (tr, 1H, phenyl-H); 7.48 (m, 1H, phenyl-H); 7.81 (tr, 1H, phenyl-H)

Compound I-8.2 (diastereomer mixture approximately: 1:1)

0.90, 0.98 (2tr, 3H, CH₃CH₂); 1.38, 1.48 (2s, 3H, CH₃); 1.70, 1.76 (2q, 2H, CH₃CH₂); 2.49 (s, 3H, CH₃Ph); 2.96 (2AB, 2H, isoxazoline-CH₂); 6.33 (s, 1H, CHCl); 7.23 (m, 1H, phenyl-H); 7.33 (m, 2H, phenyl-H); 7.78 (m, 1H, phenyl-H)

Compound I-9.2 (diastereomer mixture approximately: 1:1)

0.93, 1.00 (2tr, 3H, CH₃CH₂); 1.45, 1.49 (2s, 3H, CH₃); 1.78 (m, 2H, CH₃CH₂); 3.08 (2AB, 2H, isoxazoline-CH₂); 6.43 (2s, 1H, CHCl); 7.63 (tr, 1H, phenyl-H); 7.72 (tr, 1H, phenyl-H); 7.77 (d, 1H, phenyl-H); 8.13 (d, 1H, phenyl-H)

Compound I-42.2 (diastereomer mixture approximately: 1:1)

0.97, 1.01 (2tr, 3H, CH₃CH₂); 1.48, 1.52 (2s, 3H, CH₃); 1.88 (m, 2H, CH₃CH₂); 3.09 (2AB, 2H, isoxazoline-CH₂); 6.37 (s, 1H, CHCl); 7.15 (m, 2H, phenyl-H); 7.50 (s, 1H, phenyl-H)

Compound I-43.2 (diastereomer mixture approximately: 1:1)

0.95, 1.00 (2tr, 3H, CH₃CH₂); 1.43, 1.48 (2s, 3H, CH₃); 1.85 (m, 2H, CH₃CH₂); 3.08 (2AB, 2H, isoxazoline-CH₂); 6.62 (2s, 1H, CHCl); 7.40 (dd, 1H, phenyl-H); 7.51 (s, 1H, phenyl-H); 7.82 (d, 1H, phenyl-H)

Compound I-44.2 (diastereomer mixture approximately: 1:1)

0.89, 0.98 (2tr, 3H, CH₃CH₂); 1.37, 1.45 (2s, 3H, CH₃); 1.70, 1.77 (m, q, 2H, CH₃CH₂); 2.35, 2.44 (2s, 6H, 2CH₃Ph); 2.95 (2AB, 2H, isoxazoline-CH₂); 6.29 (s, 1H, CHCl); 7.06 (s, 1H, phenyl-H); 7.15 (d, 1H, phenyl-H); 7.68 (m, 1H, phenyl-H)

Compound I-77.2 (diastereomer mixture approximately: 1:1)

0.93, 1.00 (2tr, 3H, CH₃CH₂); 1.45, 1.49 (2s, 3H, CH₃); 1.78 (m, 2H, CH₃CH₂); 3.08 (2AB, 2H, isoxazoline-CH₂); 6.62 (2s, 1H, CHCl); 7.38 (s, 2H, phenyl-H); 7.87 (s, 1H, phenyl-H)

Compound I-78.1

m.p.: 136° C.

Compound I-78.2 (diastereomer mixture approximately: 1:1)

0.90, 0.98 (2tr, 3H, CH₃CH₂); 1.36, 1.48 (2s, 3H, CH₃); 1.69, 1.77 (m, q, 2H, CH₃CH₂); 2.38, 2.42 (2s, 6H, 2CH₃Ph); 2.96 (2AB, 2H, isoxazoline-CH₂); 6.30 (s, 1H, CHCl); 7.15 (m, 2H, phenyl-H); 7.58 (s, 1H, phenyl-H)

Compound I-109.1

m.p.: 141° C.

Compound I-109.2

0.95, 1.02 (2tr, 3H, CH₃CH₂); 1.49, 1.52 (2s, 3H, CH₃); 1.81 (m, 2H, CH₃CH₂); 3.15 (2AB, 2H, isoxazoline-CH₂); 7.28 (s, 1H, CHCl); 7.33 (m, 2H, phenyl-H); 7.42 (m, 1H, phenyl-H)

Compound I-141.2 (diastereomer mixture approximately: 1:1)

0.98, 1.02 (2tr, 3H, CH₃CH₂); 1.48, 1.51 (2s, 3H, CH₃); 1.78 (m, 2H, CH₃CH₂); 3.12 (AB, 2H, isoxazoline-CH₂); 6.32 (s, 1H, CHCl); 7.07 (dd, 1H, phenyl-H); 7.65 (m, 1H, phenyl-H);

Compound I-195.2 (diastereomer mixture approximately: 1:1)

0.73, 0.93 (2tr, 3H, CH₃CH₂); 1.19, 1.39 (2s, 3H, CH₃); 1.78 (m, 2H, CH₃CH₂); 2.78 (2AB, 2H, isoxazoline-CH₂); 6.95 (2s, 1H, CHCl); 7.52-7.65 (m, 3H, naphthyl-H); 7.91, 7.99 (2d, 2H, naphthyl-H); 8.10 (tr, 2H, naphthyl-H)

Compound I-204.2 (diastereomer mixture approximately: 1:1)

0.96, 1.00 (2tr, 3H, CH₃CH₂); 1.49, 1.45 (2s, 3H, CH₃); 1.78 (m, q, 2H, CH₃CH₂); 2.40, 2.56 (2s, 6H, 2CH₃-isoxazole); 3.10 (2AB, 2H, isoxazoline-CH₂); 5.89 (s, 1H, CHCl)

Compound I-205.1

m.p.: 122° C.

Compound I-205.2 (diastereomer mixture approximately: 1:1)

0.93, 1.00 (2tr, 3H, CH₃CH₂); 1.43, 1.49 (2s, 3H, CH₃); 1.76, 1.80 (2q, 2H, CH₃CH₂); 2.43 (s, 3H, pyrazole-CH₃); 3.09 (s, AB, 2H, isoxazoline-CH₂); 3.81 (s, 3H, NCH₃); 5.98 (s, 1H, CHCl)

Compound I-216.1

1.55 (2s, 6H, 2CH₃); 3.18 (AB, 2H, isoxazoline-CH₂); 3.96 (s, 3H, NCH₃); 6.20 (s, 1H, CHCl);

Compound I-216.2 (diastereomer mixture approximately: 1:1)

0.98 (2tr, 3H, CH₃CH₂); 1.48 (2s, 3H, CH₃); 1.81 (m, 2H, CH₃CH₂); 3.15 (2AB, 2H, isoxazoline-CH₂); 6.20 (2s, 1H, CHCl)

Compound I-244.2

0.93, 0.99 (2tr, 3H, CH₃CH₂); 1.42, 1.48 (2s, 3H, CH₃); 1.78 (m, 2H, CH₃CH₂); 3.05 (2AB, 2H, isoxazoline-CH₂); 6.09 (2s, 1H, CHCl); 7.75 (AB, 4H, phenyl-H);

Compound III-1.1 (diastereomer mixture approximately: 1:1)

Isomer A: 1.33, 1.40 (2s, 6H, 2CH₃); 2.72 (AB, 2H, isoxazoline-CH₂); 5.75 (s, 1H, CHCl); 7.42-7.56 (m, 5H, phenyl-H)

Isomer B: 1.38, 1.46 (2s, 6H, 2CH₃); 2.74 (AB, 2H, isoxazoline-CH₂); 5.58 (s, 1H, CHCl); 7.42-7.56 (m, 5H, phenyl-H)

Compound III-5.1

1.22, 1.42 (2s, 6H, 2CH₃); 3.07 (AB, 2H, isoxazoline-CH₂); 6.23 (s, 1H, CHCl); 7.10 (tr, 1H, phenyl-H); 7.25-7.50 (m, 2H, phenyl-H)

Compound III-109.1

1.57 (2s, 6H, 2CH₃); 3.11 (AB, 2H, isoxazoline-CH₂); 6.52 (s, 1H, CHCl); 7.30-7.45 (m, 3H, phenyl-H)

Compound III-109.2 (diastereomer mixture)

1.01 (2tr, 3H, CH₃CH₂); 1.50, 1.52 (2s, 3H, CH₃); 1.83 (m, 2H, CH₃CH₂); 3.19 (2AB, 2H, isoxazoline-CH₂); 6.52 (s, 1H, CHCl); 7.30-7.50 (m, 3H, phenyl-H)

Compound V-1.1

m.p.: 96° C.

Compound V-243.2

0.98 (tr, 3H, CH₃CH₂); 1.50 (s, 3H, CH₃); 1.80 (m, 2H, CH₃CH₂); 3.13 (AB, 2H, isoxazoline-CH₂); 7.67 (m, 1H, phenyl-H); 7.81 (d, 1H, phenyl-H); 8.15 (m, 2H, phenyl-H)

Compound V-244.2

m.p.: 99° C.

### B. FORMULATION EXAMPLES

a) A dust is obtained by mixing 10 parts by weight of a compound of the formula (I) and 90 parts by weight of talc as inert substance and comminuting the mixture in a hammer mill.
b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurinate as wetter and dispersant and grinding the mixture in a pinned-disk mill.
c) A dispersion concentrate which is readily dispersible in water is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.) and grinding the mixture in a ball mill to a fineness of below 5 microns.
d) An emulsifiable concentrate is obtained from 15 parts by weight of a compound of the formula (I), 75 parts by weight of cyclohexanone as the solvent and 10 parts by weight of ethoxylated nonylphenol as the emulsifier.
e) Water-dispersible granules are obtained by mixing
   75 parts by weight of a compound of the formula (I),
   10 parts by weight of calcium lignosulfonate,
   5 parts by weight of sodium lauryl sulfate,
   3 parts by weight of polyvinyl alcohol and
   7 parts by weight of kaolin,
   grinding the mixture in a pinned-disk mill and granulating the powder in a fluidized bed by spraying on water as the granulation liquid.
f) Water-dispersible granules are also obtained by homogenizing and precomminuting, in a colloid mill,
   25 parts by weight of a compound of the formula (I),
   5 parts by weight of sodium 2,2′-dinaphthylmethane-6,6′-disulfonate
   2 parts by weight of sodium oleoylmethyltaurinate,
   1 part by weight of polyvinyl alcohol,
   17 parts by weight of calcium carbonate and
   50 parts by weight of water,
   subsequently grinding the mixture in a bead mill and atomizing and drying the resulting suspension in a spray tower by means of a single-fluid nozzle.

### C. BIOLOGICAL EXAMPLES

1. Pre-Emergence Herbicidal Activity

Seeds of monocot and dicot weed and crop plants are planted out in sandy loam soil in wood fiber pots and covered with soil. The test compounds, formulated as wettable powders (WP), are then applied to the surface of the soil cover, in the form of an aqueous suspension, at a water application rate of 600 I/ha (converted), in various dosages, with the addition of 0.2% of wetting agent.

After the treatment, the pots are set up under glass and held under good growth conditions for the test plants. Visual scoring of the emergence damage to the trial plants is made after a trial period of 3 weeks in comparison with untreated controls (herbicidal activity in percent (%): 100% activity=plants have died, 0% activity=like control plants).

As the results show, compounds of the invention have good pre-emergence herbicidal activity against a broad spectrum of gramineous and broadleaf weeds. By way of example, the compounds of example I-1.1, I-2.2, I-3.2, I-4.2, I-6.2, I-8.2, I-43.2, I-78.2, III-1.1, III-5.1, III-8.2, III-109.2, V-236.2, V-244.2, VII-8, VII-9, VII-10, and also other compounds from table A, exhibit very good herbicidal activity against weed plants such as, for example, Avena spp., Lolium multiflorum, Stellaria media, Setaria spp., Sinapis alba and Amaranthus retroflexus when applied pre-emergence at a rate of 2 kg or less of active substance per hectare.

2. Post-Emergence Herbicidal Activity

Seeds of monocot and dicot weed and crop plants are planted out in sandy loam soil in wood fiber pots, covered with soil, and cultivated under glass under good growth conditions. Two-three weeks after sowing, the trial plants are treated at the one-leaf stage. The test compounds, formulated as wettable powders (WP), are then sprayed onto the green parts of the plants, at a water application rate of 600 I/ha (converted), in various dosages, with the addition of 0.2% of wetting agent. After the trial plants have stood for about 3 weeks under glass under optimum growth conditions, the effect of the products is scored visually in comparison with untreated controls. (herbicidal activity in percent (%): 100% activity=plants have died, 0% activity=like control plants).

Post-emergence too, compounds of the invention exhibit good herbicidal activity against a broad spectrum of economically important gramineous and broadleaf weeds. By way of example, the compounds of example I-1.1, I-3.2, I-6.2, I-8.2, I-43.2, I-44.2, I-78.2, I-109.2, I-141.2, III-1.1, III-5.1, III-43.2, III-109.2, V-236.2, V-244.2, VII-8, VII-9, VII-10, and also other compounds from table A, have a very good herbicidal activity against weed plants such as, for example, Sinapis alba, Echinochloa crus-galli, Cyperus iria, Avena spp., Stellaria media, Setaria spp., and Amaranthus retroflexus when applied post-emergence at a rate of 2 kg or less of active substance per hectare.

LENGTHY TABLE
The patent application contains a lengthy table section. A copy of the table is available in electronic form from the USPTO web site (http://seqdata.uspto.gov/?pageRequest=docDetail&DocID=US20070161513A1). An electronic copy of the table will also be available from the USPTO upon request and payment of the fee set forth in 37 CFR 1.19(b)(3).

1. A compound of the formula (I) or a salt thereof 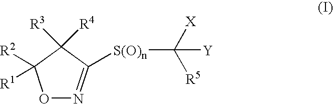
in which
R¹ and R² are each independently selected from the group consisting of H, cyano, (C₁-C₆)-alkyl, (C₂-C₆)-alkenyl, (C₂-C₈)-alkynyl, (C₃-C₆)-cycloalkyl, (C₁-C₆)-alkanoyl, (C₁-C₆)-alkoxycarbonyl, mono-((C₁-C₆)-alkyl)-aminocarbonyl, and di-((C₁-C₆)-alkyl)-aminocarbonyl, wherein each of the (C₁-C₆)-alkyl, (C₂-C₆)-alkenyl, (C₂-C₆)-alkynyl, (C₃-C₆)-cycloalkyl, (C₁-C₆)-alkanoyl, (C₁-C₆)-alkoxycarbonyl, mono-((C₁-C₆)-alkyl)-aminocarbonyl, or di-((C₁-C₆)-alkyl)-aminocarbonyl radicals is unsubstituted or substituted by one or more identical or different radicals selected from the group consisting of halogen, cyano, (C₃-C₈)-cycloalkyl, —OR⁶, and —S(O)ₘR⁶, in which m is 0, 1 or 2, R⁶ is (C₁-C₆)-alkyl, (C₂-C₆)-alkenyl, (C₂-C₆)-alkynyl or (C₃-C₆)-cycloalkyl, any of which is unsubstituted or substituted by one or more identical or different radicals selected from the group consisting of halogen and cyano,
or are each independently unsubstituted or substituted phenyl or heterocyclyl, wherein the heterocyclyl is saturated or unsaturated, and wherein each of the aforementioned aryl or heterocyclyl radicals is unsubstituted or substituted by one or more identical or different radicals selected from the group consisting of (C₁-C₆)-alkyl, (C₁-C₆)-haloalkyl, halogen, cyano, (C₃-C₈)-cycloalkyl, —OC(R⁷)₃, and —SC(R⁷)₃, wherein R⁷ is independently at each occurrence H, F or Cl,
or R¹ and R² together with the carbon atom to which they are attached form a spirolinkage of 3 to 7 carbon atoms
R³ and R⁴ are H, (C₁-C₆)-alkyl, (C₂-C₆)-alkenyl, (C₂-C₆)-alkynyl or (C₃-C₆)-cycloalkyl, wherein the (C₁-C₆)-alkyl, (C₂-C₆)-alkenyl, (C₂-C₆)-alkynyl or (C₃-C₆)-cycloalkyl are optionally substituted by one or more identical or different radicals selected from the group consisting of halogen, nitro, cyano, (C₃-C₆)-cycloalkyl, (C₁-C₆)-alkoxy, (C₁-C₆)-haloalkoxy, and (C₁-C₆)-alkylthio, or R³ and R⁴ together with the carbon atom to which they are attached form a spirolinkage of 3 to 7 carbon atoms,
or
R¹ and R³ form, together with the carbon atoms to which they are attached, a ring structure composed of 5-8 carbon atoms,
R⁵ is unsubstituted or substituted aryl, or unsubstituted or substituted heteroaryl, wherein each of the aryl or heteroaryl radicals is optionally substituted by OH, halogen, cyano, (C₁-C₆)-alkyl, (C₁-C₆)-haloalkyl, (C₂-C₆)-alkenyl, (C₂-C₆)-alkynyl, (C₃-C₆)-cycloalkyl, (C₃-C₆)-cycloalkenyl, mono-(C₁-C₆)-alkylamino, di-((C₁-C₆)-alkyl)-amino, N—((C₁-C₆)-alkanoyl)-amino, (C₁-C₆)-alkoxy, (C₁-C₆)-haloalkoxy, (C₃-C₆)-alkenyloxy, (C₃-C₆)-alkynyloxy, (C₃-C₆)-cycloalkoxy, (C₄-C₆)-cycloalkenyloxy, (C₁-C₆)-alkylthio, (C₁-C₆)-haloalkylthio, (C₃-C₆)-cycloalkylthio, (C₃-C₆)-alkenylthio, (C₄-C₆)-cycloalkenylthio, (C₃-C₆)-alkynylthio, (C₁-C₆)-alkanoyl, (C₂-C₆)-alkenylcarbonyl, (C₂-C₆)-alkynylcarbonyl, arylcarbonyl, (C₁-C₆)-alkoxycarbonyl, (C₃-C₆)-alkenyloxycarbonyl, (C₃-C₆)-alkynoxy-carbonyl, aryloxycarbonyl, (C₁-C₆)-alkylsulfinyl, (C₁-C₆)-alkyl-sulfonyl, (C₁-C₆)-haloalkylsulfinyl or (C₁-C₆)-haloalkylsulfonyl, and wherein the aforementioned alkyl, alkoxy or haloalkoxy radicals are optionally linked cyclically with one another when they are in an ortho relationship,
n is 1 or 2,
x is F, Cl, Br or I, and
Y is H, F, Cl, Br or I.

2. The compound as claimed in claim 1, wherein R¹ and R² are each independently selected from the group consisting of H, (C₁-C₄)-alkyl, (C₂-C₃)-alkenyl, (C₂-C₃)-alkynyl and (C₃-C₆)-cycloalkyl, wherein each of the (C₁-C₄)-alkyl, (C₂-C₃)-alkenyl, (C₂-C₃)-alkynyl and (C₃-C₆)-cycloalkyl radicals is optionally substituted by one or more radicals selected from the group consisting of halogen, (C₁-C₃)-alkoxy, cyano, and (C₁-C₃)-cycloalkyl.

3. The compound as claimed in claim 1, wherein R¹ and R² are each independently (C₁-C₄)-alkyl, wherein each of the (C₁-C₄)-alkyl radicals is optionally substituted by one or more identical or different halogens.

4. The compound as claimed in claim 3, wherein R¹ and R² are each independently methyl or ethyl, any of which is optionally independently monohalogenated or polyhalogenated.

5. The compound as claimed in claim 4, wherein R¹ and R² are each independently methyl, ethyl, chloromethyl or fluoromethyl.

6. The compound as claimed in claim 5, wherein R¹ and R² are each independently methyl, ethyl or chloromethyl.

7. The compound as claimed in claim 6, wherein R¹ and R² are each independently methyl or ethyl.

8. The compound as claimed in claim 1, wherein R³ and R⁴ are H or (C₁-C₄)-alkyl, wherein the alkyl is optionally substituted by one or more identical or different radicals selected from the group consisting of halogen and cyano.

9. The compound as claimed in claim 8, wherein R³ and R⁴ are H, methyl or ethyl.

10. The compound as claimed in claim 9, wherein R³ and R⁴ are H.

11. The compound as claimed in claim 1, wherein R⁵ is an unsubstituted or substituted aryl or an unsubstituted or substituted heteroaryl, wherein each of the aryl or heteroaryl radicals is optionally substituted by OH, halogen, cyano, (C₁-C₄)-alkyl, (C₁-C₄)-haloalkyl, (C₂-C₄)-alkenyl, (C₂-C₄)-alkynyl, (C₃-C₆)-cycloalkyl, (C₅-C₆)-cycloalkenyl, mono-(C₁-C₄)-alkylamino, di-((C₁-C₄)-alkyl)-amino, N—((C₁-C₄)-alkanoyl)-amino, (C₁-C₄)-alkoxy, (C₁-C₄)-haloalkoxy, (C₃-C₄)-alkenyloxy, (C₃-C₄)-alkynyloxy, (C₄-C₆)-cycloalkoxy, (C₅-C₆)-cycloalkenyloxy, (C₁-C₄)-alkylthio, (C₁-C₆)-haloalkylthio, (C₄-C₆)-cycloalkylthio, (C₃-C₄)-alkenylthio, (C₅-C₆)-cycloalkenylthio, (C₃-C₄)-alkynylthio, (C₁-C₄)-alkanoyl, (C₂-C₄)-alkenylcarbonyl, (C₂-C₄)-alkynylcarbonyl, arylcarbonyl, (C₁-C₄)-alkoxycarbonyl, (C₃-C₄)-alkenyloxycarbonyl, (C₃-C₄)-alkynoxycarbonyl, aryloxycarbonyl, (C₁-C₄)-alkylsulfinyl, (C₁-C₄)-alkylsulfonyl or (C₁-C₄)-haloalkylsulfinyl or (C₁-C₄)-haloalkylsulfonyl, and wherein the alkyl, alkoxy, and haloalkoxy radicals are optionally linked cyclically with one another when they are in an ortho relationship.

12. The compound as claimed in claim 11, wherein R⁵ is an unsubstituted or substituted aryl having 6 to 10 carbon atoms, or unsubstituted or substituted heteroaryl, having 2 to 5 carbon atoms with 1 to 3 heteroatoms selected from the group consisting of N, O, and S, wherein each of the aryl or heteroaryl radicals is optionally substituted by one or more identical or different radicals selected from the group consisting of halogen, cyano, ethyl, methyl, haloethyl, halomethyl, methoxy, ethoxy, trifluoromethylthio, trifluoromethylsulfinyl, trifluoromethylsulfonyl, halomethoxy, and haloethoxy.

13. The compound as claimed in claim 13, wherein R⁵ is a substituted or unsubstituted phenyl, pyrazolyl, pyridinyl, pyrimidinyl, pyrazinyl, pyridazinyl, thienyl, furyl, imidazolyl, triazolyl, isothiazolyl, thiazolyl or oxazolyl, optionally substituted by one or more identical or different radicals selected from the group consisting of halogen, cyano, ethyl, methyl, methoxy, ethoxy, halomethoxy, haloethoxy, trifluoromethylthio, trifluoromethylsulfinyl, trifluoromethylsulfonyl, halo ethyl, and halomethyl.

14. The compound as claimed in claim 13, wherein R⁵ is a phenyl or pyrazolyl having one, two or three identical or different substituents selected from the group consisting of fluorine, chlorine, methyl, trifluoromethyl, methoxy, difluoromethoxy, and trifluoromethoxy.

15. The compound as claimed in claim 1, wherein X is chlorine, fluorine or bromine.

16. The compound as claimed in claim 15, wherein X is chlorine or fluorine.

17. The compound as claimed in claim 1, wherein Y is H, chlorine, fluorine or bromine.

18. The compound as claimed in Claim 1, wherein X is Cl and Y is H.

19. The compound as claimed in claim 1, wherein X is F and Y is H.

20. The compound as claimed in claim 1, wherein X is Br and Y is H.

21. The compound as claimed in claim 1, wherein n is 2.

22. (canceled)

23. A method of controlling weed plants or of regulating plant growth, which comprises applying an effective amount of one or more compounds of the formula (I) or salts thereof as claimed in claim 1 to plants, parts of plants, seeds of plants or the cultivation area.

24. The method as claimed in claim 23, wherein the method comprises one or more applications of one or more compounds of the formula (I) alone or in conjunction with one or more customary plant protection formulating auxiliaries.

25. The method as claimed in claim 24, wherein two or more applications are carried out in sequence, and wherein the two or more applications optionally have different concentrations or combinations of compounds of the formula (I) or both.

26. A herbicidal or plant growth regulating composition comprising one or more compounds of the formula (I) or salts thereof as claimed in claim 1 and one or more customary plant protection formulating auxiliaries.

27. A process for preparing a compound of the formula (I′), 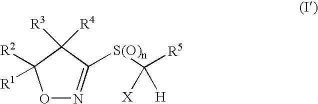
where R¹, R², R³, R⁴, and R⁵ are as defined in claim 1, n is 1 or 2, and X is fluorine, or a salt thereof, which comprises fluorinating a thioether of the formula (II) 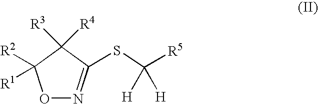
in which R¹, R², R³, R⁴, and R⁵ are as defined in claim 1
with an electrophilic fluorinating agent in the position alpha to the sulfur atom and subjecting the resulting alpha-fluorothioether of the formula (III) 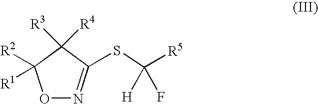
in which R¹, R², R³, R⁴, and R⁵ are as defined in claim 1
a) to oxidation with one equivalent of an oxidizing agent, to give the fluorosulfoxide (I′) wherein n is 1, or
b) to oxidation with two equivalents of an oxidizing agent, to give the fluorosulfone (I′) wherein n is 2.

28. The process as claimed in claim 27, wherein 1-chloromethyl-4-fluoro-1,4-diazabicyclo[2.2.2]octane bistetrafluoroborate is used as the fluorinating agent for preparing the fluorothioether of the formula (III).

29. The process as claimed in claim 27, wherein
a) hydrogen peroxide, sodium metaperiodate, an organic peroxide or an organic peroxy acid is used as the oxidizing agent for preparing the fluorosulfoxide, and
b) hydrogen peroxide, an organic peroxide or an organic peroxy acid is used as an oxidizing agent for preparing the fluorosulfone.

30. A compound of the formula (III) prepared by the method according to claim 27.

31. A process for preparing a compound of the formula (I″), 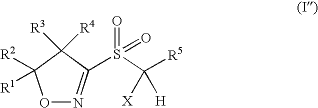
in which R¹, R², R³, R⁴, and R⁵ are as defined in claim 1 and X is fluorine, chlorine or bromine, or a salt thereof, which comprises deprotonating
a sulfone of the formula (IV), 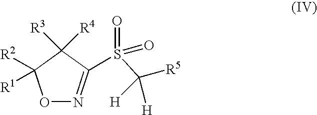
in which R¹, R², R³, R⁴, and R⁵ are as defined in claim 1
in the position alpha to the sulfur atom and subjecting the resulting carbanion alternatively to
a) fluorination with an electrophilic fluorinating agent, to give the fluorosulfone of the formula (I″) wherein X=fluorine, or
b) chlorination with a chlorinating reagent, to give the chlorosulfone of the formula (I″) wherein X=chlorine, or
c) bromination with a brominating reagent, to give the bromosulfone of the formula (I″) wherein X=bromine.

32. The process as claimed in claim 31, wherein the sulfone of the formula (IV) is deprotonated with a strong base in a suitable inert solvent and then reacted with an electrophilic fluorinating agent.

33. The process as claimed in claim 31, wherein the sulfone of the formula (IV) is deprotonated with a strong base and then
a) chlorinated with carbon tetrachloride, to give the chlorosulfone of the formula (I″) wherein X=chlorine, or
b) brominated with carbon tetrabromide, to give the bromosulfone of the formula (I″) wherein X=Br.

34. A process for preparing a compound of the formula (I′″) 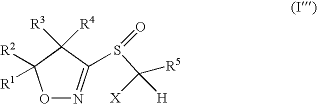
in which R¹, R², R³, R⁴, and R⁵ are as defined in claim 1 and X is chlorine, or a salt thereof, which comprises deprotonating a sulfoxide of the formula (V) 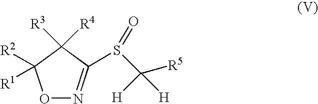
in which R¹, R², R³, R⁴, and R⁵ are as defined in claim 1
in the position alpha to the sulfur atom and reacting the resultant carbanion with a halogenating agent to give the chlorosulfoxide of the formula (I′″).

35. A process for preparing a compound of the formula (I′″), 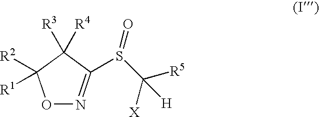
in which R¹, R², R³, R⁴, and R⁵ are as defined in claim 1 and X is chlorine, or a salt thereof, which comprises reacting a thioether of the formula (II) 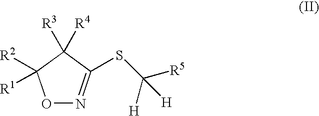
in which R¹, R², R³, R⁴, and R⁵ are as defined in claim 1
with elemental chlorine in dichloromethane/water in the presence of sodium dihydrogen phosphate.

36. A process for preparing a compound of the formula (I″″) 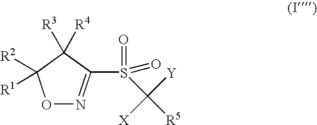
in which R¹, R², R³, R⁴, and R⁵ are as defined in claim 1 and X and Y are chlorine, which comprises deprotonating a sulfone of the formula (IV), 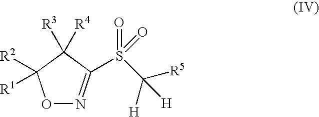
in which R¹, R², R³, R⁴, and R⁵ are as defined in claim 1, in the position alpha to the sulfur atom and subjecting the resulting carbanion to chlorination with carbon tetrachloride in excess, to give the compound of formula (I″″), wherein X and Y are Cl.

37. A process for preparing a compound of the formula (I″″) 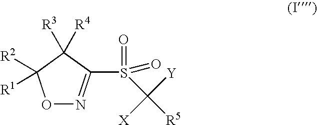
in which R¹, R², R³, R⁴, and R⁵ are as defined in claim 1 and X and Y are fluorine, which comprises deprotonating a sulfone of the formula (IV), 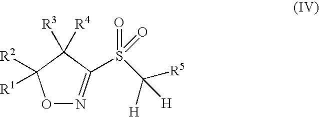
in which R¹, R², R³, R⁴, and R⁵ are as defined in claim 1, in the position alpha to the sulfur atom with a strong base and subjecting the resulting carbanion to fluorination with an electrophilic fluorinating agent, wherein twice the equivalent of the base and of the fluorinating agent are used compared to the equivalent of the base and the fluorinating agent used when preparing a monofluorosulfone compound where X is F and Y is H.

38. A process for preparing a compound of the formula (I″″) 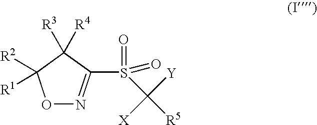
in which R¹, R², R³, R⁴, and R⁵ are as defined in claim 1 and X and Y are bromine, which comprises deprotonating a sulfone of the formula (IV), 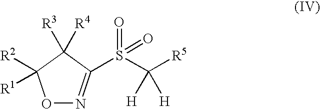
in which R¹, R², R³, R⁴, and R⁵ are as defined in claim 1, in the position alpha to the sulfur atom and subjecting the resulting carbanion to bromination with carbon tetrabromide in excess, to give the compound of formula (I″″), wherein X and Y are bromine.

39. The compound as claimed in claim 13, wherein R⁵ is a unsubstituted or substituted phenyl or pyrazolyl optionally substituted by one or more identical or different radicals selected from the group consisting of halogen, cyano, ethyl, methyl, methoxy, ethoxy, halomethoxy, haloethoxy, trifluoromethylthio, trifluoromethylsulfinyl, trifluoromethylsulfonyl, haloethyl, and halomethyl.

40. The compound as claimed in claim 14, wherein R⁵ is phenyl or pyrazolyl having one or two identical or different substituents selected from the group consisting of fluorine, chlorine, methyl, trifluoromethyl, methoxy, difluoromethoxy, and trifluoromethoxy.
